(19) United States
(12) Patent Application Publication
(10) Pub. No.: US 2022/0031815 A1
(43) Pub. Date: Feb. 3, 2022

(54) ANIMAL FEED COMPOSITION AND USE THEREOF

(71) Applicants: DSM IP ASSETS B.V., Teerlen (NL); NOVOZYMES A/S, Bagsvaerd (DK)

(72) Inventors: Javier Alejandro AMERI, Kaiseraugst (CH); Leticia CARDOSO BITTENCOURT, Kaiseraugst (CH); Marcelo HIDALGO, Kaiseraugst (CH); Rual LOPEZ-ULIBARRI, Kaiseraugst (CH); Vijay MAKHIJA, Kaiseraugst (CH); Estefania PEREZ CALVO, Kaiseraugst (CH); Rolando VALIENTES, Kaiseraugst (CH)

(21) Appl. No.: 17/275,801

(22) PCT Filed: Sep. 17, 2019
(86) PCT No.: PCT/EP2019/074773

§ 371 Date: Mar. 12, 2021

(30) Foreign Application Priority Data
Sep. 17, 2018 (EP) 18194824.1
May 28, 2019 (EP) 19176972.8

(51) Int. Cl. A61K 38/47; A23K 20/195; A23K 20/189; A23K 10/30; A23K 50/75; A61K 38/12; A61K 31/7036; A61K 31/43; A61K 31/498; A61K 31/351; A61K 31/4025; A61K 31/555; A61K 31/7048; A61K 31/444; A61K 9/00; A61P 3/02

(52) U.S. Cl. CPC A61K 38/47; A61P 3/02; A23K 20/189; A23K 10/30; A23K 50/75; A61K 38/12; A61K 31/7036; A61K 31/43; A61K 31/498; A61K 31/351; A61K 31/4025; A61K 31/555; A61K 31/7048; A61K 31/444; A61K 9/0056; A23K 20/195

(57) ABSTRACT

The present invention provides a composition and/or an animal feed comprising polypeptides having muramidase activity and uses thereof.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to a composition and/or an animal feed comprising polypeptides having muramidase activity and uses thereof.

### Description of the Related Art

Muramidase, also known as Lysozyme, is an O-glycosyl hydrolase produced as a defensive mechanism against bacteria by many organisms. The enzyme causes the hydrolysis of bacterial cell walls by cleaving the glycosidic bonds of peptidoglycan, an important structural molecule in bacteria. After having their cell walls weakened by muramidase action, bacterial cells lyse as a result of umbalanced osmotic pressure.

Muramidase naturally occurs in many organisms such as viruses, plants, insects, birds, reptiles and mammals. Muramidase has been classified into five different glycoside hydrolase (GH) families (CAZy, www.cazy.org): hen egg-white muramidase (GH22), goose egg-white muramidase (GH23), bacteriophage T4 muramidase (GH24), Sphingomonas flagellar protein (GH73) and Chalaropsis muramidases (GH25). Muramidases from the families GH23 and GH24 are primarily known from bacteriophages and have only recently been identified in fungi. The muramidase family GH25 has been found to be structurally unrelated to the other muramidase families.

Muramidase has traditionally been extracted from hen egg white due to its natural abundance and until very recently hen egg white muramidase was the only muramidase investigated for use in animal feed. Muramidase extracted from hen egg white is the primary product available on the commercial market, but does not cleave N,6-O-diacetylmuramic acid in e.g. Staphylococcus aureus cell walls and is thus unable to lyse this important human pathogen among others (Masschalck B, Deckers D, Michiels C W (2002), “Lytic and nonlytic mechanism of inactivation of gram-positive bacteria by muramidase under atmospheric and high hydrostatic pressure”, J Food Prot. 65(12):1916-23).

WO2000/21381 discloses a composition comprising at least two antimicrobial enzymes and a polyunsaturated fatty acid, wherein one of the antimicrobial enzymes was a GH22 muramidase from chicken egg white. GB2379166 discloses a composition comprising a compound that disrupts the peptidoglycan layer of bacteria and a compound that disrupts the phospholipid layer of bacteria, wherein the peptidoglycan disrupting compound was a GH22 muramidase from chicken egg white.

WO2004/026334 discloses an antimicrobial composition for suppressing the growth of enteric pathogens in the gut of livestock comprising (a) a cell wall lysing substance or its salt, (b) a antimicrobial substance, (c) a sequestering agent and (d) a lantibiotic, wherein the cell wall lysing substance or its salt is a GH22 muramidase from hen egg white.

Surprisingly, the inventors of the present invention discovered that muramidases in combination with an antibiotic provides unexpected effects in improving growth performance of animals.

### SUMMARY OF THE INVENTION

Accordingly, the present invention relates to a composition or an animal feed comprising one or more polypeptides having muramidase activity and one or more antibiotics.

The present invention further relates to a method of improving growth performance in an animal comprising administering to the animal the composition or the animal feed of the present invention.

The present invention further relates to use of the composition or the animal feed of the present invention in improving growth performance in an animal.

The present invention further relates to a method of reducing usage amount of an antibiotic in an animal comprising administering to the animal one or more polypeptides having muramidase activity.

The present invention further relates to use of one or more polypeptides having muramidase activity in reducing usage amount of an antibiotic in an animal.

### OVERVIEW OF SEQUENCE LISTING

SEQ ID NO: 1 is the mature amino acid sequence of a GH25 Muramidase from Acremonium alcalophilum as described in WO2013/076253 (SEQ ID NO: 4).

SEQ ID NO: 2 is the mature amino acid sequence of a GH25 Muramidase from Acremonium alcalophilum as described in WO2013/076253 (SEQ ID NO: 8).

SEQ ID NO: 3 is the mature amino acid sequence of a GH25 Muramidase from Aspergillus fumigatus as described in WO2011/104339 (SEQ ID NO: 3).

SEQ ID NO: 4 is the mature amino acid sequence of a GH25 Muramidase from Trichoderma reesei as described in WO2009/102755 (SEQ ID NO: 4).

SEQ ID NO: 5 is the mature amino acid sequence of a GH25 Muramidase from Trametes cinnabarina as described in WO2005/080559 (SEQ ID NO: 2).

SEQ ID NO: 6 is the mature amino acid sequence of a GH25 Muramidase from Sporormia fimetaria as described in PCT/CN2017/075978 (SEQ ID NO: 3).

SEQ ID NO: 7 is the mature amino acid sequence of a GH25 Muramidase from Poronia punctata as described in PCT/CN2017/075978 (SEQ ID NO: 6).

SEQ ID NO: 8 is the mature amino acid sequence of a GH25 Muramidase from Poronia punctata as described in PCT/CN2017/075978 (SEQ ID NO: 9).

SEQ ID NO: 9 is the mature amino acid sequence of a GH25 Muramidase from Lecanicillium sp. WMM742 as described in PCT/CN2017/075978 (SEQ ID NO: 12).

SEQ ID NO: 10 is the mature amino acid sequence of a GH25 Muramidase from Lecanicillium sp. WMM742 as described in PCT/CN2017/075978 (SEQ ID NO: 15).

SEQ ID NO: 11 is the mature amino acid sequence of a GH25 Muramidase from Onygena equina as described in PCT/CN2017/075978 (SEQ ID NO: 18).

SEQ ID NO: 12 is the mature amino acid sequence of a GH25 Muramidase from Purpureocillium lilacinum as described in PCT/CN2017/075978 (SEQ ID NO: 21).

SEQ ID NO: 13 is the mature amino acid sequence of a GH25 Muramidase from Trichobolus zukalii as described in PCT/CN2017/075978 (SEQ ID NO: 24).

SEQ ID NO: 14 is the mature amino acid sequence of a GH25 Muramidase from Penicillium citrinum as described in PCT/CN2017/075978 (SEQ ID NO: 27).

SEQ ID NO: 15 is the mature amino acid sequence of a GH25 Muramidase from Cladorrhinum bulbillosum as described in PCT/CN2017/075978 (SEQ ID NO: 30).

SEQ ID NO: 16 is the mature amino acid sequence of a GH25 Muramidase from Umbelopsis westeae as described in PCT/CN2017/075978 (SEQ ID NO: 33).

SEQ ID NO: 17 is the mature amino acid sequence of a GH25 Muramidase from Zygomycetes sp. XZ2655 as described in PCT/CN2017/075978 (SEQ ID NO: 36).

SEQ ID NO: 18 is the mature amino acid sequence of a GH25 Muramidase from Chaetomium cupreum as described in PCT/CN2017/075978 (SEQ ID NO: 39).

SEQ ID NO: 19 is the mature amino acid sequence of a GH25 Muramidase from Cordyceps cardinalis as described in PCT/CN2017/075978 (SEQ ID NO: 42).

SEQ ID NO: 20 is the mature amino acid sequence of a GH25 Muramidase from Penicillium sp. ‘qii’ as described in PCT/CN2017/075978 (SEQ ID NO: 45).

SEQ ID NO: 21 is the mature amino acid sequence of a GH25 Muramidase from Aspergillus sp. nov XZ2609 as described in PCT/CN2017/075978 (SEQ ID NO: 48).

SEQ ID NO: 22 is the mature amino acid sequence of a GH25 Muramidase from Paecilomyces sp. XZ2658 as described in PCT/CN2017/075978 (SEQ ID NO: 51).

SEQ ID NO: 23 is the mature amino acid sequence of a GH25 Muramidase from Paecilomyces sp. XZ2658 as described in PCT/CN2017/075978 (SEQ ID NO: 54).

SEQ ID NO: 24 is the mature amino acid sequence of a GH25 Muramidase from Pycnidiophora cf dispera as described in PCT/CN2017/075978 (SEQ ID NO: 60).

SEQ ID NO: 25 is the mature amino acid sequence of a GH25 Muramidase from Thermomucor indicae-seudaticae as described in PCT/CN2017/075978 (SEQ ID NO: 63).

SEQ ID NO: 26 is the mature amino acid sequence of a GH25 Muramidase from Isaria farinosa as described in PCT/CN2017/075978 (SEQ ID NO: 66).

SEQ ID NO: 27 is the mature amino acid sequence of a GH25 Muramidase from Lecanicillium sp. WMM742 as described in PCT/CN2017/075978 (SEQ ID NO: 69).

SEQ ID NO: 28 is the mature amino acid sequence of a GH25 Muramidase from Zopfiella sp. t180-6 as described in PCT/CN2017/075978 (SEQ ID NO: 72).

SEQ ID NO: 29 is the mature amino acid sequence of a GH25 Muramidase from Malbranchea flava as described in PCT/CN2017/075978 (SEQ ID NO: 75).

SEQ ID NO: 30 is the mature amino acid sequence of a GH25 Muramidase from Hypholoma polytrichi as described in PCT/CN2017/075978 (SEQ ID NO: 80).

SEQ ID NO: 31 is the mature amino acid sequence of a GH25 Muramidase from Aspergillus deflectus as described in PCT/CN2017/075978 (SEQ ID NO: 83).

SEQ ID NO: 32 is the mature amino acid sequence of a GH25 Muramidase from Ascobolus stictoideus as described in PCT/CN2017/075978 (SEQ ID NO: 86).

SEQ ID NO: 33 is the mature amino acid sequence of a GH25 Muramidase from Coniochaeta sp. as described in PCT/CN2017/075978 (SEQ ID NO: 89).

SEQ ID NO: 34 is the mature amino acid sequence of a GH25 Muramidase from Daldinia fissa as described in PCT/CN2017/075978 (SEQ ID NO: 92).

SEQ ID NO: 35 is the mature amino acid sequence of a GH25 Muramidase from Rosellinia sp. as described in PCT/CN2017/075978 (SEQ ID NO: 95).

SEQ ID NO: 36 is the mature amino acid sequence of a GH25 Muramidase from Ascobolus sp. ZY179 as described in PCT/CN2017/075978 (SEQ ID NO: 98).

SEQ ID NO: 37 is the mature amino acid sequence of a GH25 Muramidase from Curreya sp. XZ2623 as described in PCT/CN2017/075978 (SEQ ID NO: 101).

SEQ ID NO: 38 is the mature amino acid sequence of a GH25 Muramidase from Coniothyrium sp. as described in PCT/CN2017/075978 (SEQ ID NO: 104).

SEQ ID NO: 39 is the mature amino acid sequence of a GH25 Muramidase from Hypoxylon sp. as described in PCT/CN2017/075978 (SEQ ID NO: 107).

SEQ ID NO: 40 is the mature amino acid sequence of a GH25 Muramidase from Xylariaceae sp. 1653h as described in PCT/CN2017/075978 (SEQ ID NO: 110).

SEQ ID NO: 41 is the mature amino acid sequence of a GH25 Muramidase from Hypoxylon sp. as described in PCT/CN2017/075978 (SEQ ID NO: 113).

SEQ ID NO: 42 is the mature amino acid sequence of a GH25 Muramidase from Yunnania penicillata as described in PCT/CN2017/075978 (SEQ ID NO: 116).

SEQ ID NO: 43 is the mature amino acid sequence of a GH25 Muramidase from Engyodontium album as described in PCT/CN2017/075978 (SEQ ID NO: 119).

SEQ ID NO: 44 is the mature amino acid sequence of a GH25 Muramidase from Metapochonia bulbillosa as described in PCT/CN2017/075978 (SEQ ID NO: 122).

SEQ ID NO: 45 is the mature amino acid sequence of a GH25 Muramidase from Hamigera paravellanea as described in PCT/CN2017/075978 (SEQ ID NO: 125).

SEQ ID NO: 46 is the mature amino acid sequence of a GH25 Muramidase from Metarhizium iadini as described in PCT/CN2017/075978 (SEQ ID NO: 128).

SEQ ID NO: 47 is the mature amino acid sequence of a GH25 Muramidase from Thermoascus aurantiacus as described in PCT/CN2017/075978 (SEQ ID NO: 131).

SEQ ID NO: 48 is the mature amino acid sequence of a GH25 Muramidase from Clonostachys rossmaniae as described in PCT/CN2017/075978 (SEQ ID NO: 134).

SEQ ID NO: 49 is the mature amino acid sequence of a GH25 Muramidase from Simplicillium obclavatum as described in PCT/CN2017/075978 (SEQ ID NO: 137).

SEQ ID NO: 50 is the mature amino acid sequence of a GH25 Muramidase from Aspergillus inflatus as described in PCT/CN2017/075978 (SEQ ID NO: 140).

SEQ ID NO: 51 is the mature amino acid sequence of a GH25 Muramidase from Paracremonium inflatum as described in PCT/CN2017/075978 (SEQ ID NO: 143).

SEQ ID NO: 52 is the mature amino acid sequence of a GH25 Muramidase from Westerdykella sp. as described in PCT/CN2017/075978 (SEQ ID NO: 146).

SEQ ID NO: 53 is the mature amino acid sequence of a GH25 Muramidase from Stropharia semiglobata as described in PCT/CN2017/075978 (SEQ ID NO: 155).

SEQ ID NO: 54 is the mature amino acid sequence of a GH25 Muramidase from Gelasinospora cratophora as described in PCT/CN2017/075978 (SEQ ID NO: 158).

SEQ ID NO: 55 is the mature amino acid sequence of a GH25 Muramidase from Flammulina velutipes as described in PCT/CN2017/075978 (SEQ ID NO: 221).

SEQ ID NO: 56 is the mature amino acid sequence of a GH25 Muramidase from Deconica coprophila as described in PCT/CN2017/075978 (SEQ ID NO: 224).

SEQ ID NO: 57 is the mature amino acid sequence of a GH25 Muramidase from Rhizomucor pusillus as described in PCT/CN2017/075978 (SEQ ID NO: 227).

SEQ ID NO: 58 is the mature amino acid sequence of a GH25 Muramidase from Stropharia semiglobata as described in PCT/CN2017/075978 (SEQ ID NO: 230).

SEQ ID NO: 59 is the mature amino acid sequence of a GH25 Muramidase from Stropharia semiglobata as described in PCT/CN2017/075978 (SEQ ID NO: 233).

SEQ ID NO: 60 is the mature amino acid sequence of a GH25 Muramidase from Myceliophthora fergusii as described in PCT/CN2017/075960 (SEQ ID NO: 3).

SEQ ID NO: 61 is the mature amino acid sequence of a GH25 Muramidase from Mortierella alpina as described in PCT/CN2017/075960 (SEQ ID NO: 15).

SEQ ID NO: 62 is the mature amino acid sequence of a GH25 Muramidase from Penicillium atrovenetum as described in PCT/CN2017/075960 (SEQ ID NO: 27).

SEQ ID NO: 63 is the mature amino acid sequence of a GH24 Muramidase from Trichophaea saccata as described in WO2017/000922 (SEQ ID NO: 257).

SEQ ID NO: 64 is the mature amino acid sequence of a GH24 Muramidase from Chaetomium thermophilum as described in WO2017/000922 (SEQ ID NO: 264).

SEQ ID NO: 65 is the mature amino acid sequence of a GH24 Muramidase from Trichoderma harzianum as described in WO2017/000922 (SEQ ID NO: 267).

SEQ ID NO: 66 is the mature amino acid sequence of a GH24 Muramidase from Trichophaea minuta as described in WO2017/000922 (SEQ ID NO: 291).

SEQ ID NO: 67 is the mature amino acid sequence of a GH24 Muramidase from Chaetomium sp. ZY287 as described in WO2017/000922 (SEQ ID NO: 294).

SEQ ID NO: 68 is the mature amino acid sequence of a GH24 Muramidase from Mortierella sp. ZY002 as described in WO2017/000922 (SEQ ID NO: 297).

SEQ ID NO: 69 is the mature amino acid sequence of a GH24 Muramidase from Metarhizium sp. XZ2431 as described in WO2017/000922 (SEQ ID NO: 300).

SEQ ID NO: 70 is the mature amino acid sequence of a GH24 Muramidase from Geomyces auratus as described in WO2017/000922 (SEQ ID NO: 303).

SEQ ID NO: 71 is the mature amino acid sequence of a GH24 Muramidase from Ilyonectria rufa as described in WO2017/000922 (SEQ ID NO: 306).

### Definitions

Animal: The term “animal” refers to any animal except humans. Examples of animals are monogastric animals, including but not limited to pigs or swine (including, but not limited to, piglets, growing pigs, and sows); poultry such as turkeys, ducks, quail, guinea fowl, geese, pigeons (including squabs) and chicken (including but not limited to broiler chickens (referred to herein as broiles), chicks, layer hens (referred to herein as layers)); pets such as cats and dogs; horses (including but not limited to hotbloods, coldbloods and warm bloods) crustaceans (including but not limited to shrimps and prawns) and fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish).

Animal feed: The term “animal feed” refers to any compound, preparation, or mixture suitable for, or intended for intake by an animal. Animal feed for a monogastric animal typically comprises concentrates as well as vitamins, minerals, enzymes, direct fed microbial, amino acids and/or other feed ingredients (such as in a premix) whereas animal feed for ruminants generally comprises forage (including roughage and silage) and may further comprise concentrates as well as vitamins, minerals, enzymes direct fed microbial, amino acid and/or other feed ingredients (such as in a premix).

Concentrates: The term “concentrates” means feed with high protein and energy concentrations, such as fish meal, molasses, oligosaccharides, sorghum, seeds and grains (either whole or prepared by crushing, milling, etc. from e.g. corn, oats, rye, barley, wheat), oilseed press cake (e.g. from cottonseed, safflower, sunflower, soybean (such as soybean meal), rapeseed/canola, peanut or groundnut), palm kernel cake, yeast derived material and distillers grains (such as wet distillers grains (WDS) and dried distillers grains with solubles (DDGS)).

Forage: The term “forage” as defined herein also includes roughage. Forage is fresh plant material such as hay and silage from forage plants, grass and other forage plants, seaweed, sprouted grains and legumes, or any combination thereof. Examples of forage plants are Alfalfa (lucerne), birdsfoot trefoil, brassica (e.g. kale, rapeseed (canola), rutabaga (swede), turnip), clover (e.g. alsike clover, red clover, subterranean clover, white clover), grass (e.g. Bermuda grass, brome, false oat grass, fescue, heath grass, meadow grasses, orchard grass, ryegrass, Timothy-grass), corn (maize), millet, barley, oats, rye, sorghum, soybeans and wheat and vegetables such as beets. Forage further includes crop residues from grain production (such as corn stover; straw from wheat, barley, oat, rye and other grains); residues from vegetables like beet tops; residues from oilseed production like stems and leaves form soy beans, rapeseed and other legumes; and fractions from the refining of grains for animal or human consumption or from fuel production or other industries.

Fragment: The term “fragment” means a polypeptide or a catalytic domain having one or more (e.g., several) amino acids absent from the amino and/or carboxyl terminus of a mature polypeptide or domain; wherein the fragment has Muramidase activity.

In one aspect, a fragment of a GH24 Muramidase (such as one of SEQ ID NO: 63 to 71) comprises at least 230 amino acids, such as at least 235 amino acids, at least 240 amino acids, or at least 245 amino acids and has Muramidase activity. In another aspect, a fragment of a GH24 Muramidase (such as one of SEQ ID NO: 63 to 71) comprises at least 90% of the length of the mature polypeptide, such as at least 92%, at least 94%, at least 96%, at least 98% or at least 99% of the length of the mature polypeptide and has Muramidase activity.

In one aspect, a fragment of a GH25 Muramidase (such as one of SEQ ID NO: 1 to 72) comprises at least 180 amino acids, such as at least 185 amino acids, at least 190 amino acids, at least 195 amino acids, at least 200 amino acids, at least 205 amino acids or at least 210 amino acids and has Muramidase activity. In another aspect, a fragment of a GH25 Muramidase (such as one of SEQ ID NO: 1 to 72) comprises at least 90% of the length of the mature polypeptide, such as at least 92%, at least 94%, at least 96%, at least 98% or at least 99% of the length of the mature polypeptide and has Muramidase activity.

Muramidase activity: The term “Muramidase activity” means the enzymatic hydrolysis of the 1,4-beta-linkages between N-acetylmuramic acid and N-acetyl-D-glucosamine residues in a peptidoglycan or between N-acetyl-D-glucosamine residues in chitodextrins, resulting in bacteriolysis due to osmotic pressure. Muramidase belongs to the enzyme class EC 3.2.1.17. Muramidase activity is typically measured by turbidimetric determination. The method is based on the changes in turbidity of a suspension of Micrococcus luteus ATCC 4698 induced by the lytic action of Muramidase. In appropriate experimental conditions these changes are proportional to the amount of Muramidase in the medium (c.f. INS 1105 of the Combined Compendium of Food Additive Specifications of the Food and Agriculture Organisation of the UN (www.fao.org)). For the purpose of the present invention, Muramidase activity is determined according to the turbidity assay described in example 1 (“Determination of Muramidase Activity”) and the polypeptide has Muramidase activity if it shows activity against one or more bacteria, such as Micrococcus luteus ATCC 4698 and/or Exiguobacterium undea (DSM14481). As an example, the GH25 Muramidase of the present invention has at least 20%, e.g., at least 40%, at least 50%, at least 60%, at least 70%, at least 80%, at least 90%, at least 95%, or at least 100% of the Muramidase activity of SEQ ID NO: 1. As another example, the GH24 Muramidase of the present invention have at least 20%, e.g., at least 40%, at least 50%, at least 60%, at least 70%, at least 80%, at least 90%, at least 95%, or at least 100% of the Muramidase activity of SEQ ID NO: 63.

Mature polypeptide: The term “mature polypeptide” means a polypeptide in its final form following translation and any post-translational modifications, such as N-terminal processing, C-terminal truncation, glycosylation, phosphorylation, etc.

In the present invention, the mature polypeptide may be amino acids 1 to 208 of SEQ ID NO: 1, amino acids 1 to 213 of SEQ ID NO: 2, amino acids 1 to 218 of SEQ ID NO: 3, amino acids 1 to 208 of SEQ ID NO: 4, amino acids 1 to 215 of SEQ ID NO: 5, amino acids 1 to 207 of SEQ ID NO: 6, amino acids 1 to 201 of SEQ ID NO: 7, amino acids 1 to 201 of SEQ ID NO: 8, amino acids 1 to 203 of SEQ ID NO: 9, amino acids 1 to 208 of SEQ ID NO: 10, amino acids 1 to 207 of SEQ ID NO: 11, amino acids 1 to 208 of SEQ ID NO: 12, amino acids 1 to 207 of SEQ ID NO: 13, amino acids 1 to 207 of SEQ ID NO: 14, amino acids 1 to 207 of SEQ ID NO: 15, amino acids 1 to 208 of SEQ ID NO: 16, amino acids 1 to 208 of SEQ ID NO: 17, amino acids 1 to 206 of SEQ ID NO: 18, amino acids 1 to 207 of SEQ ID NO: 19, amino acids 1 to 216 of SEQ ID NO: 20, amino acids 1 to 218 of SEQ ID NO: 21, amino acids 1 to 204 of SEQ ID NO: 22, amino acids 1 to 203 of SEQ ID NO: 23, amino acids 1 to 208 of SEQ ID NO: 24, amino acids 1 to 210 of SEQ ID NO: 25, amino acids 1 to 207 of SEQ ID NO: 26, amino acids 1 to 207 of SEQ ID NO: 27, amino acids 1 to 208 of SEQ ID NO: 28, amino acids 1 to 217 of SEQ ID NO: 29, amino acids 1 to 208 of SEQ ID NO: 30, amino acids 1 to 201 of SEQ ID NO: 31, amino acids 1 to 202 of SEQ ID NO: 32, amino acids 1 to 207 of SEQ ID NO: 33, amino acids 1 to 202 of SEQ ID NO: 34, amino acids 1 to 201 of SEQ ID NO: 35, amino acids 1 to 202 of SEQ ID NO: 36, amino acids 1 to 206 of SEQ ID NO: 37, amino acids 1 to 202 of SEQ ID NO: 38, amino acids 1 to 202 of SEQ ID NO: 39, amino acids 1 to 202 of SEQ ID NO: 40, amino acids 1 to 202 of SEQ ID NO: 41, amino acids 1 to 206 of SEQ ID NO: 42, amino acids 1 to 207 of SEQ ID NO: 43, amino acids 1 to 208 of SEQ ID NO: 44, amino acids 1 to 215 of SEQ ID NO: 45, amino acids 1 to 217 of SEQ ID NO: 46, amino acids 1 to 214 of SEQ ID NO: 47, amino acids 1 to 208 of SEQ ID NO: 48, amino acids 1 to 203 of SEQ ID NO: 49, amino acids 1 to 216 of SEQ ID NO: 50, amino acids 1 to 207 of SEQ ID NO: 51, amino acids 1 to 208 of SEQ ID NO: 52, amino acids 1 to 207 of SEQ ID NO: 53, amino acids 1 to 208 of SEQ ID NO: 54, amino acids 1 to 207 of SEQ ID NO: 55, amino acids 1 to 207 of SEQ ID NO: 56, amino acids 1 to 208 of SEQ ID NO: 57, amino acids 1 to 207 of SEQ ID NO: 58, amino acids 1 to 207 of SEQ ID NO: 59, amino acids 1 to 207 of SEQ ID NO: 60, amino acids 1 to 204 of SEQ ID NO: 61, amino acids 1 to 216 of SEQ ID NO: 62, amino acids 1 to 245 of SEQ ID NO: 63, amino acids 1 to 249 of SEQ ID NO: 64, amino acids 1 to 248 of SEQ ID NO: 65, amino acids 1 to 245 of SEQ ID NO: 66, amino acids 1 to 249 of SEQ ID NO: 67, amino acids 1 to 245 of SEQ ID NO: 68, amino acids 1 to 247 of SEQ ID NO: 69, amino acids 1 to 250 of SEQ ID NO: 70, amino acids 1 to 240 of SEQ ID NO: 71.

Obtained or obtainable from: The term “obtained or obtainable from” means that the polypeptide may be found in an organism from a specific taxonomic rank. Preferably, the polypeptide is obtained or obtainable from the kingdom Fungi, wherein the term kingdom is the taxonomic rank. More preferably, the polypeptide is obtained or obtainable from the phylum Ascomycota, wherein the term phylum is the taxonomic rank. More preferably, the polypeptide is obtained or obtainable from the subphylum Pezizomycotina, wherein the term subphylum is the taxonomic rank. More preferably, the polypeptide is obtained or obtainable from the class Eurotiomycetes, wherein the term class is the taxonomic rank.

If the taxonomic rank of a polypeptide is not known, it can easily be determined by a person skilled in the art by performing a BLASTP search of the polypeptide (using e.g. the National Center for Biotechnology Information (NCIB) website http://www.ncbi.nlm.nih.gov/) and comparing it to the closest homologues. The skilled person can also compare the sequence to those of the application as filed. An unknown polypeptide which is a fragment of a known polypeptide is considered to be of the same taxonomic species. An unknown natural polypeptide or artificial variant which comprises a substitution, deletion and/or insertion in up to 10 positions is considered to be from the same taxonomic species as the known polypeptide.

Roughage: The term “roughage” means dry plant material with high levels of fiber, such as fiber, bran, husks from seeds and grains and crop residues (such as stover, copra, straw, chaff, sugar beet waste).

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

Variant: The term “variant” means a polypeptide having Muramidase activity comprising an alteration, i.e., a substitution, insertion, and/or deletion, of one or more (several) amino acid residues at one or more (e.g., several) positions. A substitution means replacement of the amino acid occupying a position with a different amino acid; a deletion means removal of the amino acid occupying a position; and an insertion means adding 1, 2, or 3 amino acids adjacent to and immediately following the amino acid occupying the position.

In the present invention, a Muramidase variant may comprise from 1 to 10 alterations, i.e. 1, 2, 3, 4, 5, 6, 7, 8, 9 or 10 alterations and have at least 20%, e.g., at least 40%, at least 50%, at least 60%, at least 70%, at least 80%, at least 90%, at least 95%, or at least 100% of the Muramidase activity of the parent Muramidase, such as SEQ ID NO: 1 or SEQ ID NO: 63.

Antibiotic: The term “antibiotic” or “antibiotics” means a synthetic or naturally-derived organic chemical substance, used most often at low concentrations, in the treatment of infectious diseases of animals, which prevents or inhibits the growth of microorganisms.

### DETAILED DESCRIPTION OF THE INVENTION

### Composition

It has been surprisingly found that a composition comprising a muramidase (preferably a fungal muramidase) and an antibiotic gives an additional performance benefit in animals.

Thus, in a first aspect, the invention relates to a composition comprising one or more polypeptides having muramidase activity and one or more antibiotics.

In the present invention, the polypeptide having muramidase activity may be a GH24 muramidase, preferably a fungal GH24 muramidase, preferably obtained or obtainable from the phylum Ascomycota, more preferably from the class Eurotiomycetes. the polypeptide having muramidase activity may also be a GH25 muramidase, preferably a fungal GH25 muramidase, preferably obtained or obtainable from the phylum Ascomycota, more preferably from the class Eurotiomycetes.

In the present invention, the antibiotics may be any one of Bacitracin, Bambermycin, Carbadox, Enramycin, Enduracidin, Laidlomycin, Lasalocid, Lincomycin, Monensin, Neomycin, Penicillin, Roxarsone, Roxarsone, Salinomycin, Tylosin, and Virginiamycin.

Preferably, the invention relates to a composition comprising one or more polypeptides having muramidase activity and one or more antibiotics, wherein the polypeptide having muramidase activity is selected from the group consisting of:
(a) a polypeptide having at least 80%, e.g., at least 85%, at least 90%, at least 95%, or 100% sequence identity to SEQ ID NO: 1;
(b) a polypeptide having at least 80%, e.g., at least 85%, at least 90%, at least 95%, or 100% sequence identity to SEQ ID NO: 2;
(c) a polypeptide having at least 80%, e.g., at least 85%, at least 90%, at least 95%, or 100% sequence identity to SEQ ID NO: 3;
(d) a polypeptide having at least 80%, e.g., at least 85%, at least 90%, at least 95%, or 100% sequence identity to SEQ ID NO: 4;
(e) a polypeptide having at least 80%, e.g., at least 85%, at least 90%, at least 95%, or 100% sequence identity to SEQ ID NO: 5;
(f) a polypeptide having at least 80%, e.g., at least 85%, at least 90%, at least 95%, or 100% sequence identity to SEQ ID NO: 6;
(g) a polypeptide having at least 80%, e.g., at least 85%, at least 90%, at least 95%, or 100% sequence identity to SEQ ID NO: 7;
(h) a polypeptide having at least 80%, e.g., at least 85%, at least 90%, at least 95%, or 100% sequence identity to SEQ ID NO: 8;
(i) a polypeptide having at least 80%, e.g., at least 85%, at least 90%, at least 95%, or 100% sequence identity to SEQ ID NO: 9;
(j) a polypeptide having at least 80%, e.g., at least 85%, at least 90%, at least 95%, or 100% sequence identity to SEQ ID NO: 10;
(k) a polypeptide having at least 80%, e.g., at least 85%, at least 90%, at least 95%, or 100% sequence identity to SEQ ID NO: 11;
(l) a polypeptide having at least 80%, e.g., at least 85%, at least 90%, at least 95%, or 100% sequence identity to SEQ ID NO: 12;
(m) a polypeptide having at least 80%, e.g., at least 85%, at least 90%, at least 95%, or 100% sequence identity to SEQ ID NO: 13;
(n) a polypeptide having at least 80%, e.g., at least 85%, at least 90%, at least 95%, or 100% sequence identity to SEQ ID NO: 14;
(o) a polypeptide having at least 80%, e.g., at least 85%, at least 90%, at least 95%, or 100% sequence identity to SEQ ID NO: 15;
(p) a polypeptide having at least 80%, e.g., at least 85%, at least 90%, at least 95%, or 100% sequence identity to SEQ ID NO: 16;
(q) a polypeptide having at least 80%, e.g., at least 85%, at least 90%, at least 95%, or 100% sequence identity to SEQ ID NO: 17;
(r) a polypeptide having at least 80%, e.g., at least 85%, at least 90%, at least 95%, or 100% sequence identity to SEQ ID NO: 18;
(s) a polypeptide having at least 80%, e.g., at least 85%, at least 90%, at least 95%, or 100% sequence identity to SEQ ID NO: 19;
(t) a polypeptide having at least 80%, e.g., at least 85%, at least 90%, at least 95%, or 100% sequence identity to SEQ ID NO: 20;
(u) a polypeptide having at least 80%, e.g., at least 85%, at least 90%, at least 95%, or 100% sequence identity to SEQ ID NO: 21;
(v) a polypeptide having at least 80%, e.g., at least 85%, at least 90%, at least 95%, or 100% sequence identity to SEQ ID NO: 22;
(w) a polypeptide having at least 80%, e.g., at least 85%, at least 90%, at least 95%, or 100% sequence identity to SEQ ID NO: 23;
(x) a polypeptide having at least 80%, e.g., at least 85%, at least 90%, at least 95%, or 100% sequence identity to SEQ ID NO: 24;
(y) a polypeptide having at least 80%, e.g., at least 85%, at least 90%, at least 95%, or 100% sequence identity to SEQ ID NO: 25;
(z) a polypeptide having at least 80%, e.g., at least 85%, at least 90%, at least 95%, or 100% sequence identity to SEQ ID NO: 26;
(aa) a polypeptide having at least 80%, e.g., at least 85%, at least 90%, at least 95%, or 100% sequence identity to SEQ ID NO: 27;
(ab) a polypeptide having at least 80%, e.g., at least 85%, at least 90%, at least 95%, or 100% sequence identity to SEQ ID NO: 28;
(ac) a polypeptide having at least 80%, e.g., at least 85%, at least 90%, at least 95%, or 100% sequence identity to SEQ ID NO: 29;
(ad) a polypeptide having at least 80%, e.g., at least 85%, at least 90%, at least 95%, or 100% sequence identity to SEQ ID NO: 30;
(ae) a polypeptide having at least 80%, e.g., at least 85%, at least 90%, at least 95%, or 100% sequence identity to SEQ ID NO: 31;
(af) a polypeptide having at least 80%, e.g., at least 85%, at least 90%, at least 95%, or 100% sequence identity to SEQ ID NO: 32;

(ag) a polypeptide having at least 80%, e.g., at least 85%, at least 90%, at least 95%, or 100% sequence identity to SEQ ID NO: 33;
(ah) a polypeptide having at least 80%, e.g., at least 85%, at least 90%, at least 95%, or 100% sequence identity to SEQ ID NO: 34;
(ai) a polypeptide having at least 80%, e.g., at least 85%, at least 90%, at least 95%, or 100% sequence identity to SEQ ID NO: 35;
(aj) a polypeptide having at least 80%, e.g., at least 85%, at least 90%, at least 95%, or 100% sequence identity to SEQ ID NO: 36;
(ak) a polypeptide having at least 80%, e.g., at least 85%, at least 90%, at least 95%, or 100% sequence identity to SEQ ID NO: 37;
(al) a polypeptide having at least 80%, e.g., at least 85%, at least 90%, at least 95%, or 100% sequence identity to SEQ ID NO: 38;
(am) a polypeptide having at least 80%, e.g., at least 85%, at least 90%, at least 95%, or 100% sequence identity to SEQ ID NO: 39;
(an) a polypeptide having at least 80%, e.g., at least 85%, at least 90%, at least 95%, or 100% sequence identity to SEQ ID NO: 40;
(ao) a polypeptide having at least 80%, e.g., at least 85%, at least 90%, at least 95%, or 100% sequence identity to SEQ ID NO: 41;
(ap) a polypeptide having at least 80%, e.g., at least 85%, at least 90%, at least 95%, or 100% sequence identity to SEQ ID NO: 42;
(aq) a polypeptide having at least 80%, e.g., at least 85%, at least 90%, at least 95%, or 100% sequence identity to SEQ ID NO: 43;
(ar) a polypeptide having at least 80%, e.g., at least 85%, at least 90%, at least 95%, or 100% sequence identity to SEQ ID NO: 44;
(as) a polypeptide having at least 80%, e.g., at least 85%, at least 90%, at least 95%, or 100% sequence identity to SEQ ID NO: 45;
(at) a polypeptide having at least 80%, e.g., at least 85%, at least 90%, at least 95%, or 100% sequence identity to SEQ ID NO: 46;
(au) a polypeptide having at least 80%, e.g., at least 85%, at least 90%, at least 95%, or 100% sequence identity to SEQ ID NO: 47;
(av) a polypeptide having at least 80%, e.g., at least 85%, at least 90%, at least 95%, or 100% sequence identity to SEQ ID NO: 48;
(aw) a polypeptide having at least 80%, e.g., at least 85%, at least 90%, at least 95%, or 100% sequence identity to SEQ ID NO: 49;
(ax) a polypeptide having at least 80%, e.g., at least 85%, at least 90%, at least 95%, or 100% sequence identity to SEQ ID NO: 50;
(ay) a polypeptide having at least 80%, e.g., at least 85%, at least 90%, at least 95%, or 100% sequence identity to SEQ ID NO: 51;
(az) a polypeptide having at least 80%, e.g., at least 85%, at least 90%, at least 95%, or 100% sequence identity to SEQ ID NO: 52;
(ba) a polypeptide having at least 80%, e.g., at least 85%, at least 90%, at least 95%, or 100% sequence identity to SEQ ID NO: 53;
(bb) a polypeptide having at least 80%, e.g., at least 85%, at least 90%, at least 95%, or 100% sequence identity to SEQ ID NO: 54;
(bc) a polypeptide having at least 80%, e.g., at least 85%, at least 90%, at least 95%, or 100% sequence identity to SEQ ID NO: 55;
(bd) a polypeptide having at least 80%, e.g., at least 85%, at least 90%, at least 95%, or 100% sequence identity to SEQ ID NO: 56;
(be) a polypeptide having at least 80%, e.g., at least 85%, at least 90%, at least 95%, or 100% sequence identity to SEQ ID NO: 57;
(bf) a polypeptide having at least 80%, e.g., at least 85%, at least 90%, at least 95%, or 100% sequence identity to SEQ ID NO: 58;
(bg) a polypeptide having at least 80%, e.g., at least 85%, at least 90%, at least 95%, or 100% sequence identity to SEQ ID NO: 59;
(bh) a polypeptide having at least 80%, e.g., at least 85%, at least 90%, at least 95%, or 100% sequence identity to SEQ ID NO: 60;
(bi) a polypeptide having at least 80%, e.g., at least 85%, at least 90%, at least 95%, or 100% sequence identity to SEQ ID NO: 61;
(bj) a polypeptide having at least 80%, e.g., at least 85%, at least 90%, at least 95%, or 100% sequence identity to SEQ ID NO: 62;
(bk) a polypeptide having at least 80%, e.g., at least 85%, at least 90%, at least 95%, or 100% sequence identity to SEQ ID NO: 63;
(bl) a polypeptide having at least 80%, e.g., at least 85%, at least 90%, at least 95%, or 100% sequence identity to SEQ ID NO: 64;
(bm) a polypeptide having at least 80%, e.g., at least 85%, at least 90%, at least 95%, or 100% sequence identity to SEQ ID NO: 65;
(bn) a polypeptide having at least 80%, e.g., at least 85%, at least 90%, at least 95%, or 100% sequence identity to SEQ ID NO: 66;
(bo) a polypeptide having at least 80%, e.g., at least 85%, at least 90%, at least 95%, or 100% sequence identity to SEQ ID NO: 67;
(bp) a polypeptide having at least 80%, e.g., at least 85%, at least 90%, at least 95%, or 100% sequence identity to SEQ ID NO: 68;
(bq) a polypeptide having at least 80%, e.g., at least 85%, at least 90%, at least 95%, or 100% sequence identity to SEQ ID NO: 69;
(br) a polypeptide having at least 80%, e.g., at least 85%, at least 90%, at least 95%, or 100% sequence identity to SEQ ID NO: 70;
(bs) a polypeptide having at least 80%, e.g., at least 85%, at least 90%, at least 95%, or 100% sequence identity to SEQ ID NO: 71;
(bt) a variant of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70 or SEQ ID NO: 71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions;
(bu) a polypeptide comprising the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), (bs) or (bt) and a N-terminal and/or C-terminal extension of between 1 and 10 amino acids; and
(bv) a fragment of a polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), (bs) or (bt) having Muramidase activity and having at least 90% of the length of the mature polypeptide.

More preferably, the polypeptide having muramidase activity comprises or consists of amino acids 1 to 208 of SEQ ID NO: 1, amino acids 1 to 213 of SEQ ID NO: 2, amino acids 1 to 218 of SEQ ID NO: 3, amino acids 1 to 208 of SEQ ID NO: 4, amino acids 1 to 215 of SEQ ID NO: 5, amino acids 1 to 207 of SEQ ID NO: 6, amino acids 1 to 201 of SEQ ID NO: 7, amino acids 1 to 201 of SEQ ID NO: 8, amino acids 1 to 203 of SEQ ID NO: 9, amino acids 1 to 208 of SEQ ID NO: 10, amino acids 1 to 207 of SEQ ID NO: 11, amino acids 1 to 208 of SEQ ID NO: 12, amino acids 1 to 207 of SEQ ID NO: 13, amino acids 1 to 207 of SEQ ID NO: 14, amino acids 1 to 207 of SEQ ID NO: 15, amino acids 1 to 208 of SEQ ID NO: 16, amino acids 1 to 208 of SEQ ID NO: 17, amino acids 1 to 206 of SEQ ID NO: 18, amino acids 1 to 207 of SEQ ID NO: 19, amino acids 1 to 216 of SEQ ID NO: 20, amino acids 1 to 218 of SEQ ID NO: 21, amino acids 1 to 204 of SEQ ID NO: 22, amino acids 1 to 203 of SEQ ID NO: 23, amino acids 1 to 208 of SEQ ID NO: 24, amino acids 1 to 210 of SEQ ID NO: 25, amino acids 1 to 207 of SEQ ID NO: 26, amino acids 1 to 207 of SEQ ID NO: 27, amino acids 1 to 208 of SEQ ID NO: 28, amino acids 1 to 217 of SEQ ID NO: 29, amino acids 1 to 208 of SEQ ID NO: 30, amino acids 1 to 201 of SEQ ID NO: 31, amino acids 1 to 202 of SEQ ID NO: 32, amino acids 1 to 207 of SEQ ID NO: 33, amino acids 1 to 202 of SEQ ID NO: 34, amino acids 1 to 201 of SEQ ID NO: 35, amino acids 1 to 202 of SEQ ID NO: 36, amino acids 1 to 206 of SEQ ID NO: 37, amino acids 1 to 202 of SEQ ID NO: 38, amino acids 1 to 202 of SEQ ID NO: 39, amino acids 1 to 202 of SEQ ID NO: 40, amino acids 1 to 202 of SEQ ID NO: 41, amino acids 1 to 206 of SEQ ID NO: 42, amino acids 1 to 207 of SEQ ID NO: 43, amino acids 1 to 208 of SEQ ID NO: 44, amino acids 1 to 215 of SEQ ID NO: 45, amino acids 1 to 217 of SEQ ID NO: 46, amino acids 1 to 214 of SEQ ID NO: 47, amino acids 1 to 208 of SEQ ID NO: 48, amino acids 1 to 203 of SEQ ID NO: 49, amino acids 1 to 216 of SEQ ID NO: 50, amino acids 1 to 207 of SEQ ID NO: 51, amino acids 1 to 208 of SEQ ID NO: 52, amino acids 1 to 207 of SEQ ID NO: 53, amino acids 1 to 208 of SEQ ID NO: 54, amino acids 1 to 207 of SEQ ID NO: 55, amino acids 1 to 207 of SEQ ID NO: 56, amino acids 1 to 208 of SEQ ID NO: 57, amino acids 1 to 207 of SEQ ID NO: 58, amino acids 1 to 207 of SEQ ID NO: 59, amino acids 1 to 207 of SEQ ID NO: 60, amino acids 1 to 204 of SEQ ID NO: 61, amino acids 1 to 216 of SEQ ID NO: 62, amino acids 1 to 245 of SEQ ID NO: 63, amino acids 1 to 249 of SEQ ID NO: 64, amino acids 1 to 248 of SEQ ID NO: 65, amino acids 1 to 245 of SEQ ID NO: 66, amino acids 1 to 249 of SEQ ID NO: 67, amino acids 1 to 245 of SEQ ID NO: 68, amino acids 1 to 247 of SEQ ID NO: 69, amino acids 1 to 250 of SEQ ID NO: 70 or amino acids 1 to 240 of SEQ ID NO: 71.

More preferably, the composition of the present invention comprises the muramidase of SEQ ID NO: 1 and Enramycin or Bacitracin.

In the present invention, the polypeptide having muramidase activity may be dosed between 10 mg to 1000 mg enzyme protein per kg animal feed, such as 50 mg to 900 mg, 100 mg to 800 mg, 200 mg to 700 mg, 300 mg to 600 mg, or 400 mg to 500 mg enzyme protein per kg animal feed, or any combination of these intervals.

Preferably, the antibiotics may be dosed between 0.1 mg to 100 mg per kg animal feed, such as 0.5 mg to 90 mg, 1 mg to 80 mg, 5 mg to 70 mg, 10 mg to 60 mg, 15 mg to 50 mg or 20 mg to 40 mg per kg animal feed, or any combination of these intervals.

In the present invention, the polypeptide having muramidase activity and/or the antibiotic of the composition may be formulated as a solid formulation or a liquid formulation.

In the present invention, the liquid formulation may further comprise polyol which is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600.

In the present invention, the liquid formulation may further comprise preservative, preferably selected from the group consisting of sodium sorbate, potassium sorbate, sodium benzoate and potassion benzoate or any combination thereof.

In the present invention, the liquid formulation may comprise one or more formulating agents (such as those described herein), preferably a formulating agent selected from the list consisting of glycerol, ethylene glycol, 1,2-propylene glycol or 1,3-propylene glycol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, PVA, acetate and phosphate, preferably selected from the list consisting of 1,2-propylene glycol, 1,3-propylene glycol, sodium sulfate, dextrin, cellulose, sodium thiosulfate, kaolin and calcium carbonate.

In the present invention, the solid formulation may be for example as a granule, spray dried powder or agglomerate (e.g. as disclosed in WO2000/70034). The formulating agent may comprise a salt (organic or inorganic zinc, sodium, potassium or calcium salts such as e.g. such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as e.g. sucrose, dextrin, glucose, lactose, sorbitol).

Preferably, the formulating agents of the solid formulation are selected from the list consisting of sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch and cellulose. Preferably, the formulating agent is selected from one or more of the following compounds: sodium sulfate, dextrin, cellulose, sodium thiosulfate, magnesium sulfate and calcium carbonate.

In the present invention, the composition may further comprise one or more carriers. The carrier may be selected from the group consisting of water, glycerol, ethylene glycol, 1,2-propylene glycol or 1,3-propylene glycol, sodium chloride, sodium benzoate, potassium sorbate, sodium sulfate, potassium sulfate, magnesium sulfate, sodium thiosulfate, calcium carbonate, sodium citrate, dextrin, maltodextrin, glucose, sucrose, sorbitol, lactose, wheat flour, wheat bran, corn gluten meal, starch, kaolin and cellulose or any combination thereof.

Animal Feed

In the second aspect, the present invention relates to an animal feed comprising an animal feed additive, one or more protein sources and one or more energy sources characterised in that the animal feed further comprises one or more polypeptides having muramidase activity and one or more antibiotics as defined above.

Animal feed compositions or diets have a relatively high content of protein. Poultry and pig diets can be characterised as indicated in Table B of WO 01/58275, columns 2-3. Fish diets can be characterised as indicated in column 4 of this Table B. Furthermore, such fish diets usually have a crude fat content of 200-310 g/kg.

An animal feed composition according to the invention has a crude protein content of 50-800 g/kg. The protein source may be vegetable protein source and/or animal protein.

The vegetable proteins may be derived from vegetable protein sources, such as legumes and cereals, for example, materials from plants of the families Fabaceae (Leguminosae), Cruciferaceae, Chenopodiaceae, and Poaceae, such as soy bean meal, lupin meal, rapeseed meal, and combinations thereof. The protein content of the vegetable proteins is at least 10, 20, 30, 40, 50, 60, 70, 80, or 90% (w/w).

Preferably, the vegetable protein source may be material from one or more plants of the family Fabaceae, e.g., soybean, lupine, pea, or bean. The vegetable protein source may also be material from one or more plants of the family Chenopodiaceae, e.g. beet, sugar beet, spinach or quinoa. Other examples of vegetable protein sources are rapeseed, and cabbage. In the present invention, soybean is a preferred vegetable protein source. Other examples of vegetable protein sources are cereals such as barley, wheat, rye, oat, maize (corn), rice, and sorghum.

Besides the vegetable protein as defined above, the animal feed of the invention may also contain animal protein, such as Meat and Bone Meal, Feather meal, and/or Fish Meal, typically in an amount of 0-25%. The animal feed of the invention may also comprise Dried Distillers Grains with Solubles (DDGS), typically in amounts of 0-30%.

Preferably, the protein source is selected from the group consisting of soybean, wild soybean, beans, lupin, tepary bean, scarlet runner bean, slimjim bean, lima bean, French bean, Broad bean (fava bean), chickpea, lentil, peanut, Spanish peanut, canola, sunflower seed, cotton seed, rapeseed (oilseed rape) or pea or in a processed form such as soybean meal, full fat soy bean meal, soy protein concentrate (SPC), fermented soybean meal (FSBM), sunflower meal, cotton seed meal, rapeseed meal, fish meal, bone meal, feather meal, whey or any combination thereof.

Furthermore, or in the alternative (to the crude protein content indicated above), the animal feed composition of the invention may have a content of metabolisable energy of 10-30 MJ/kg. In present invention, the energy source may be selected from the group consisting of maize, corn, sorghum, barley, wheat, oats, rice, triticale, rye, beet, sugar beet, spinach, potato, cassava, quinoa, cabbage, switchgrass, millet, pearl millet, foxtail millet or in a processed form such as milled corn, milled maize, potato starch, cassava starch, milled sorghum, milled switchgrass, milled millet, milled foxtail millet, milled pearl millet, or any combination thereof.

Furthermore, or in the alternative (to the crude protein content indicated above), the animal feed composition of the invention may have a content of calcium of 0.1-200 g/kg; and/or a content of available phosphorus of 0.1-200 g/kg; and/or a content of methionine of 0.1-100 g/kg; and/or a content of methionine plus cysteine of 0.1-150 g/kg; and/or a content of lysine of 0.5-50 g/kg.

In particular, the content of metabolisable energy, crude protein, calcium, phosphorus, methionine, methionine plus cysteine, and/or lysine may be within any one of ranges 2, 3, 4 or 5 in Table B of WO 01/58275 (R. 2-5).

Crude protein is calculated as nitrogen (N) multiplied by a factor 6.25, i.e. Crude protein (g/kg)=N (g/kg)×6.25. The nitrogen content is determined by the Kjeldahl method (A.O.A.C., 1984, Official Methods of Analysis 14th ed., Association of Official Analytical Chemists, Washington D.C.).

Metabolisable energy can be calculated on the basis of the NRC publication Nutrient requirements in swine, ninth revised edition 1988, subcommittee on swine nutrition, committee on animal nutrition, board of agriculture, national research council. National Academy Press, Washington, D.C., pp. 2-6, and the European Table of Energy Values for Poultry Feed-stuffs, Spelderholt centre for poultry research and extension, 7361 DA Beekbergen, The Netherlands. Grafisch bedrijf Ponsen & Iooijen by, Wageningen. ISBN 90-71463-12-5.

The dietary content of calcium, available phosphorus and amino acids in complete animal diets is calculated on the basis of feed tables such as Veevoedertabel 1997, gegevens over chemische samenstelling, verteerbaarheid en voederwaarde van voedermiddelen, Central Veevoederbureau, Runderweg 6, 8219 pk Lelystad. ISBN 90-72839-13-7.

Preferably, the animal feed of the invention contains 0-80% maize; and/or 0-80% sorghum; and/or 0-70% wheat; and/or 0-70% Barley; and/or 0-30% oats; and/or 0-40% soybean meal; and/or 0-25% fish meal; and/or 0-25% meat and bone meal; and/or 0-20% whey.

The final muramidase concentration in the feed is within the range of 10 mg to 1000 mg enzyme protein per kg animal feed, such as 50 mg to 900 mg, 100 mg to 800 mg, 200 mg to 700 mg, 300 mg to 600 mg, or 400 mg to 500 mg enzyme protein per kg animal feed, or any combination of these intervals.

The final antibiotic concentration in the feed is within the range of between 0.1 mg to 100 mg per kg animal feed, such as 0.5 mg to 90 mg, 1 mg to 80 mg, 5 mg to 70 mg, 10 mg to 60 mg, 15 mg to 50 mg or 20 mg to 40 mg per kg animal feed, or any combination of these intervals.

In present invention, the animal feed may further comprise one or more additional enzymes; one or more eubiotics; one or more vitamins; one or more minerals, one or more amino acids, and one or more other feed ingredients, as described below.

### Additional Enzymes

In the present invention, the compositions and/or the animal feed described herein may optionally include one or more enzymes. Enzymes can be classified on the basis of the handbook Enzyme Nomenclature from NC-IUBMB, 1992), see also the ENZYME site at the internet: http://www.expasy.ch/enzyme/.

Another classification of certain glycoside hydrolase enzymes, such as endoglucanase, galactanase, mannanase, dextranase, and galactosidase is described in Henrissat et al, “The carbohydrate-active enzymes database (CAZy) in 2013”, Nucl. Acids Res. (1 Jan. 2014) 42 (D1): D490-D495; see also www.cazy.org.

Thus the composition, the animal feed or the animal feed additive of the present invention may also comprise at least one other enzyme selected from the group comprising of acetylxylan esterase (EC 3.1.1.23), acylglycerol lipase (EC 3.1.1.72), alpha-amylase (EC 3.2.1.1), beta-amylase (EC 3.2.1.2), arabinofuranosidase (EC 3.2.1.55), cellobiohydrolases (EC 3.2.1.91), cellulase (EC 3.2.1.4), feruloyl esterase (EC 3.1.1.73), galactanase (EC 3.2.1.89), alpha-galactosidase (EC 3.2.1.22), beta-galactosidase (EC 3.2.1.23), beta-glucanase (EC 3.2.1.6), beta-glucosidase (EC 3.2.1.21), triacylglycerol lipase (EC 3.1.1.3), lysophospholipase (EC 3.1.1.5), Muramidase (EC 3.2.1.17), alpha-mannosidase (EC 3.2.1.24), beta-mannosidase (mannanase) (EC 3.2.1.25), phytase (EC 3.1.3.8, EC 3.1.3.26, EC 3.1.3.72), phospholipase A1 (EC 3.1.1.32), phospholipase A2 (EC 3.1.1.4), phospholipase D (EC 3.1.4.4), protease (EC 3.4), pullulanase (EC 3.2.1.41), pectinesterase (EC 3.1.1.11), xylanase (EC 3.2.1.8, EC 3.2.1.136), beta-xylosidase (EC 3.2.1.37), or any combination thereof.

The composition, the animal feed or the animal feed additive of the invention may also comprise a galactanase (EC 3.2.1.89) and a beta-galactosidase (EC 3.2.1.23).

The composition, the animal feed or the animal feed additive of the present invention may also comprise a phytase (EC 3.1.3.8 or 3.1.3.26). Examples of commercially available phytases include Bio-Feed™ Phytase (Novozymes), Ronozyme® P, Ronozyme® NP and Ronozyme® HiPhos (DSM Nutritional Products), Natuphos™ (BASF), Natuphos' E (BASF), Finase® and Quantum® Blue (AB Enzymes), OptiPhos® (Huvepharma), AveMix® Phytase (Aveve Biochem), Phyzyme® XP (Verenium/DuPont) and Axtra® PHY (DuPont). Other preferred phytases include those described in e.g. WO 98/28408, WO 00/43503, and WO 03/066847.

The composition, the animal feed or the animal feed additive of the present invention may also comprise a xylanase (EC 3.2.1.8). Examples of commercially available xylanases include Ronozyme® WX (DSM Nutritional Products), Econase® XT and Barley (AB Vista), Xylathin® (Verenium), Hostazym® X (Huvepharma), Axtra® XB (Xylanase/beta-glucanase, DuPont) and Axtra® XAP (Xylanase/amylase/protease, DuPont), AveMix® XG 10 (xylanase/glucanase) and AveMix® 02 CS (xylanase/glucanase/pectinase, Aveve Biochem), and Naturgrain (BASF).

The composition, the animal feed or the animal feed additive of the invention may also comprise a protease (EC 3.4). Examples of commercially available proteases include Ronozyme® ProAct (DSM Nutritional Products), Winzyme Pro Plus® (Suntaq International Limited) and Cibenza® DP100 (Novus International).

The composition, the animal feed or the animal feed additive of the invention may also comprise an alpha-amylase (EC 3.2.1.1). Examples of commercially available alpha-amylases include Ronozyme® A and RONOZYME® RumiStar™ (DSM Nutritional Products).

The composition, the animal feed or the animal feed additive of the invention may also comprise a multicomponent enzyme product, such as FRA® Octazyme (Framelco), Ronozyme® G2, Ronozyme® VP and Ronozyme® MultiGrain (DSM Nutritional Products), Rovabio® Excel or Rovabio® Advance (Adisseo).

### Eubiotics

The composition, the animal feed or the animal feed additive of the invention may additionally comprise eubiotics. Eubiotics are compounds which are designed to give a healthy balance of the micro-flora in the gastrointestinal tract. Eubiotics cover a number of different feed additives, such as probiotics, prebiotics, phytogenics (essential oils) and organic acids which are described in more detail below.

### Probiotics

In the present invention, the composition, the animal feed or the animal feed additive may further comprise one or more additional probiotic. In particular, the animal feed composition may further comprise a bacterium from one or more of the following genera: Lactobacillus, Lactococcus, Streptococcus, Bacillus, Pediococcus, Enterococcus, Leuconostoc, Carnobacterium, Propionibacterium, Bifidobacterium, Clostridium and Megasphaera or any combination thereof.

Preferably, the composition, the animal feed or the animal feed additive further comprises a bacterium from one or more of the following strains: Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Enterococcus faecium, Enterococcus spp, and Pediococcus spp, Lactobacillus spp, Bifidobacterium spp, Lactobacillus acidophilus, Pediococsus acidilactici, Lactococcus lactis, Bifidobacterium bifidum, Propionibacterium thoenii, Lactobacillus farciminus, Lactobacillus rhamnosus, Clostridium butyricum, Bifidobacterium animalis ssp. animalis, Lactobacillus reuteri, Lactobacillus salivarius ssp. salivarius, Megasphaera elsdenii, Propionibacteria sp.

More preferably, the composition or the animal feed of the present invention further comprises a bacterium from one or more of the following strains of Bacillus subtilis: 3A-P4 (PTA-6506), 15A-P4 (PTA-6507), 22C-P1 (PTA-6508), 2084 (NRRL B-500130), LSSA01 (NRRL-B-50104), BS27 (NRRL B-501 05), BS 18 (NRRL B-50633), BS 278 (NRRL B-50634), DSM 29870, DSM 29871, DSM 32315, NRRL B-50136, NRRL B-50605, NRRL B-50606, NRRL B-50622 and PTA-7547.

More preferably, the composition or the animal feed of the present invention further comprises a bacterium from one or more of the following strains of Bacillus pumilus: NRRL B-50016, ATCC 700385, NRRL B-50885 or NRRL B-50886.

More preferably, the composition or the animal feed of the present invention further comprises a bacterium from one or more of the following strains of Bacillus lichenformis: NRRL B 50015, NRRL

B-50621 or NRRL B-50623.

More preferably, the composition or the animal feed of the present invention further comprises a bacterium from one or more of the following strains of Bacillus amyloliquefaciens: DSM 29869, DSM 29869, NRRL B 50607, PTA-7543, PTA-7549, NRRL B-50349, NRRL B-50606, NRRL B-50013, NRRL B-50151, NRRL B-50141, NRRL B-50147 or NRRL B-50888.

The bacterial count of each of the bacterial strains in the composition, the animal feed or the animal feed additive is between 1×10⁴ and 1×10¹⁴ CFU/kg of dry matter, preferably between 1×10⁶ and 1×10¹² CFU/kg of dry matter, and more preferably between 1×10⁷ and 1×10¹¹ CFU/kg of dry matter. Preferably, the bacterial count of each of the bacterial strains in the composition, the animal feed or the animal feed additive is between 1×10⁸ and 1×10¹⁰ CFU/kg of dry matter.

The bacterial count of each of the bacterial strains in the composition, the animal feed or the animal feed additive is between 1×10⁵ and 1×10¹⁵ CFU/animal/day, preferably between 1×10⁷ and 1×10¹³ CFU/animal/day, and more preferably between 1×10⁸ and 1×10¹² CFU/animal/day. Preferably, the bacterial count of each of the bacterial strains in the composition, the animal feed or the animal feed additive is between 1×10⁹ and 1×10″ CFU/animal/day. More preferably, the amount of probiotics is 0.001% to 10% by weight of the composition or the animal feed or animal feed additive.

In the present invention, the one or more bacterial strains may be present in the form of a stable spore.

Examples of commercial products are Cylactin® (DSM Nutritional Products), Alterion (Adisseo), Enviva PRO (DuPont Animal Nutrition), Syncra® (mix enzyme+probiotic, DuPont Animal Nutrition), Ecobiol® and Fecinor® (Norel/Evonik) and GutCare® PY1 (Evonik).

### Prebiotics

Prebiotics are substances that induce the growth or activity of microorganisms (e.g., bacteria and fungi) that contribute to the well-being of their host. Prebiotics are typically non-digestible fiber compounds that pass undigested through the upper part of the gastrointestinal tract and stimulate the growth or activity of advantageous bacteria that colonize the large bowel by acting as substrate for them. Normally, prebiotics increase the number or activity of bifidobacteria and lactic acid bacteria in the GI tract.

Yeast derivatives (inactivated whole yeasts or yeast cell walls) can also be considered as prebiotics. They often comprise mannan-oligosaccharids, yeast beta-glucans or protein contents and are normally derived from the cell wall of the yeast, Saccharomyces cerevisiae.

In the present invention, the amount of prebiotics may be 0.001% to 10% by weight of the composition. Examples of yeast products are Yang® and Agrimos (Lallemand Animal Nutrition).

### Phytogenics

Phytogenics are a group of natural growth promoters or non-antibiotic growth promoters used as feed additives, derived from herbs, spices or other plants. Phytogenics can be single substances prepared from essential oils/extracts, essential oils/extracts, single plants and mixture of plants (herbal products) or mixture of essential oils/extracts/plants (specialized products).

Examples of phytogenics are rosemary, sage, oregano, thyme, clove, and lemongrass. Examples of essential oils are thymol, eugenol, meta-cresol, vaniline, salicylate, resorcine, guajacol, gingerol, lavender oil, ionones, irone, eucalyptol, menthol, peppermint oil, alpha-pinene; limonene, anethol, linalool, methyl dihydrojasmonate, carvacrol, propionic acid/propionate, acetic acid/acetate, butyric acid/butyrate, rosemary oil, clove oil, geraniol, terpineol, citronellol, amyl and/or benzyl salicylate, cinnamaldehyde, plant polyphenol (tannin), turmeric and curcuma extract.

In the present invention, the amount of phytogeneics may be 0.001% to 10% by weight of the composition. Examples of commercial products are Crina® (DSM Nutritional Products); Cinergy™, Biacid™, ProHacid™ Classic and ProHacid™ Advance™ (all Promivi/Cargill) and Envivo EO (DuPont Animal Nutrition).

### Organic Acids

Organic acids (C1-C7) are widely distributed in nature as normal constituents of plants or animal tissues. They are also formed through microbial fermentation of carbohydrates mainly in the large intestine. They are often used in swine and poultry production as a replacement of antibiotic growth promoters since they have a preventive effect on the intestinal problems like necrotic enteritis in chickens and Escherichia coli infection in young pigs. Organic acids can be sold as mono component or mixtures of typically 2 or 3 different organic acids. Examples of organic acids are short chain fatty acids (e.g. formic acid, acetic acid, propionic acid, butyric acid), medium chain fatty acids (e.g. caproic acid, caprylic acid, capric acid, lauric acid), di/tri-carboxylic acids (e.g. fumaric acid), hydroxy acids (e.g. lactic acid), aromatic acids (e.g. benzoic acid), citric acid, sorbic acid, malic acid, and tartaric acid or their salt (typically sodium or potassium salt such as potassium diformate or sodium butyrate).

In the present invention, the amount of organic acid may be 0.001% to 10% by weight of the composition. Examples of commercial products are VevoVitall® (DSM Nutritional Products), Amasil®, Luprisil®, Lupro-Grain®, Lupro-Cid®, Lupro-Mix® (BASF), n-Butyric Acid AF (OXEA) and Adimix Precision (Nutriad).

### Amino Acids

The composition or the animal feed of the invention may further comprise one or more amino acids. Examples of amino acids which are used are lysine, alanine, beta-alanine, threonine, methionine and tryptophan. In the present invention, the amount of amino acid may be 0.001% to 10% by weight of the composition or the animal feed.

### Vitamins and Minerals

In the present invention, the composition or the animal feed may include one or more vitamins, such as one or more fat-soluble vitamins and/or one or more water-soluble vitamins. In addition, the composition or the animal feed may optionally include one or more minerals, such as one or more trace minerals and/or one or more macro minerals.

Usually fat- and water-soluble vitamins, as well as trace minerals form part of a so-called premix intended for addition to the feed, whereas macro minerals are usually separately added to the feed.

Non-limiting examples of fat-soluble vitamins include vitamin A, vitamin D3, vitamin E, and vitamin K, e.g., vitamin K3.

Non-limiting examples of water-soluble vitamins include vitamin C, vitamin B12, biotin and choline, vitamin B1, vitamin B2, vitamin B6, niacin, folic acid and panthothenate, e.g., Ca-D-panthothenate.

Non-limiting examples of trace minerals include boron, cobalt, chloride, chromium, copper, fluoride, iodine, iron, manganese, molybdenum, iodine, selenium and zinc.

Non-limiting examples of macro minerals include calcium, magnesium, phosphorus, potassium and sodium.

In the present invention, the amount of vitamins may be 0.001% to 10% by weight of the composition or the animal feed. Preferably, the amount of minerals is 0.001% to 10% by weight of the composition or the animal feed.

The nutritional requirements of these components (exemplified with poultry and piglets/pigs) are listed in Table A of WO 01/58275. Nutritional requirement means that these components should be provided in the diet in the concentrations indicated.

In the alternative, the composition or the animal feed of the invention comprises at least one of the individual components specified in Table A of WO 01/58275. At least one means either of, one or more of, one, or two, or three, or four and so forth up to all thirteen, or up to all fifteen individual components. More specifically, this at least one individual component is included in the additive of the invention in such an amount as to provide an in-feed-concentration within the range indicated in column four, or column five, or column six of Table A.

Preferably, the composition or the animal feed of the invention comprises at least one of the below vitamins, preferably to provide an in-feed-concentration within the ranges specified in the below Table 1 (for piglet diets, and broiler diets, respectively).

TABLE 1
Typical vitamin recommendations

| Vitamin | Piglet diet | | Broiler diet | |
|---|---|---|---|---|
| Vitamin A | 10,000-15,000 | IU/kg feed | 8-12,500 | IU/kg feed |
| Vitamin D3 | 1800-2000 | IU/kg feed | 3000-5000 | IU/kg feed |
| Vitamin E | 60-100 | mg/kg feed | 150-240 | mg/kg feed |
| Vitamin K3 | 2-4 | mg/kg feed | 2-4 | mg/kg feed |
| Vitamin B1 | 2-4 | mg/kg feed | 2-3 | mg/kg feed |
| Vitamin B2 | 6-10 | mg/kg feed | 7-9 | mg/kg feed |
| Vitamin B6 | 4-8 | mg/kg feed | 3-6 | mg/kg feed |
| Vitamin B12 | 0.03-0.05 | mg/kg feed | 0.015-0.04 | mg/kg feed |
| Niacin | 30-50 | mg/kg feed | 50-80 | mg/kg feed |
| (Vitamin B3) | | | | |
| Pantothenic | 20-40 | mg/kg feed | 10-18 | mg/kg feed |
| acid | | | | |
| Folic acid | 1-2 | mg/kg feed | 1-2 | mg/kg feed |
| Biotin | 0.15-0.4 | mg/kg feed | 0.15-0.3 | mg/kg feed |
| Choline | 200-400 | mg/kg feed | 300-600 | mg/kg feed |
| chloride | | | | |

### Other Feed Ingredients

The composition or the animal feed of the invention may further comprise colouring agents, stabilisers, growth improving additives and aroma compounds/flavourings, polyunsaturated fatty acids (PUFAs); reactive oxygen generating species, antioxidants, anti-microbial peptides, anti-fungal polypeptides and mycotoxin management compounds.

Examples of colouring agents are carotenoids such as beta-carotene, astaxanthin, and lutein.

Examples of aroma compounds/flavourings are creosol, anethol, deca-, undeca- and/or dodeca-lactones, ionones, irone, gingerol, piperidine, propylidene phatalide, butylidene phatalide, capsaicin and tannin.

Examples of antimicrobial peptides (AMP's) are CAP18, Leucocin A, Tritrpticin, Protegrin-1, Thanatin, Defensin, Lactoferrin, Lactoferricin, and Ovispirin such as Novispirin (Robert Lehrer, 2000), Plectasins, and Statins, including the compounds and polypeptides disclosed in WO 03/044049 and WO 03/048148, as well as variants or fragments of the above that retain antimicrobial activity.

Examples of antifungal polypeptides (AFP's) are the Aspergillus giganteus, and Aspergillus niger peptides, as well as variants and fragments thereof which retain antifungal activity, as disclosed in WO 94/01459 and WO 02/090384.

Examples of polyunsaturated fatty acids are C18, C20 and C22 polyunsaturated fatty acids, such as arachidonic acid, docosohexaenoic acid, eicosapentaenoic acid and gamma-linoleic acid.

Examples of reactive oxygen generating species are chemicals such as perborate, persulphate, or percarbonate; and enzymes such as an oxidase, an oxygenase or a syntethase.

Antioxidants can be used to limit the number of reactive oxygen species which can be generated such that the level of reactive oxygen species is in balance with antioxidants.

Mycotoxins, such as deoxynivalenol, aflatoxin, zearalenone and fumonisin can be found in animal feed and can result in negative animal performance or illness. Compounds which can manage the levels of mycotoxin, such as via deactivation of the mycotoxin or via binding of the mycotoxin, can be added to the feed to ameliorate these negative effects. Examples of mycotoxin management compounds are Vitafix®, Vitafix Ultra (Nuscience), Mycofix®, Mycofix® Secure, FUMzyme®, Biomin® BBSH, Biomin® MTV (Biomin), Mold-Nil®, Toxy-Nil® and Unike® Plus (Nutriad).

Methods of Improving Animal Performance and/or Reducing Usage Amount of an Antibiotic

In the third aspect, the invention further relates to a method for improving growth performance in an animal comprising administering to the animal the composition or the animal feed comprising one or more polypeptides having muramidase activity and one or more antibiotics as defined above.

In the fourth aspect, the invention further relates to a method for reducing usage amount of an antibiotic in an animal comprising administering to the animal one or more polypeptides having muramidase activity.

Particularly, the invention relates to a method for improving Body Weight (BW), Body Weight Gain (BWG), Feed Conversion Ratio (FCR) and European Production Efficiency Factor (EPEF) of an animal comprising administering to the animal the composition or the animal feed comprising one or more polypeptides having muramidase activity and one or more antibiotics as defined above.

In the present invention, the improvement means increase of BW, BWG or EPEF, or decrease of FCR, compared to the same feed but excluding the polypeptide having muramidase activity or the antibiotics or both.

In the present invention, BW, BWG or EPEF may be increased by at least 1%, such as by at least 1.5%, at least 2.0%, at least 2.5%, at least 3%, at least 3.5%, at least 4% or at least 5%.

In the present invention, FCR may be decreased by at least 1%, such as by at least 1.5%, at least 2.0%, at least 2.5%, at least 3%, at least 3.5%, at least 4% or at least 5%.

In the present invention, the usage amount of an antibiotic in an animal are reduced by at least 10%, such as by at least 20%, at least 30%, at least 40%, at least 50%, at least 60%, at least 70%, at least 80%, at least 90% or at least 100% compared to the negative control wherein the polypeptide having muramidase activity is not present.

In the present invention, the polypeptide having muramidase activity may be dosed at a level of 10 mg to 1000 mg enzyme protein per kg animal feed, such as 50 mg to 900 mg, 100 mg to 800 mg, 200 mg to 700 mg, 300 mg to 600 mg, or 400 mg to 500 m per kg animal feed, or any combination of these intervals.

In the present invention, the antibiotic may be dosed at a level of 0.1 mg to 100 mg per kg animal feed, such as 0.5 mg to 90 mg, 1 mg to 80 mg, 5 mg to 70 mg, 10 mg to 60 mg, 15 mg to 50 mg or 20 mg to 40 mg per kg animal feed, or any combination of these intervals.

In the present invention, the animal is a mono-gastric animal, e.g. pigs or swine (including, but not limited to, piglets, growing pigs, and sows); poultry (including but not limited to poultry, turkey, duck, quail, guinea fowl, goose, pigeon, squab, chicken, broiler, layer, pullet and chick); pet animals such as cats and dogs, fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish); and crustaceans (including but not limited to shrimps and prawns). In a more preferred embodiment, the animal is selected from the group consisting of swine, poultry, crustaceans and fish. In an even more preferred embodiment, the animal is selected from the group consisting of swine, piglet, growing pig, sow, chicken, broiler, layer, pullet and chick.

Use in Improving Animal Performance and/or Reducing Usage Amount of an Antibiotic

In the fifth aspect, the invention further relates to use of a composition or an animal feed in improving growth performance and/or reducing usage amount of antibiotics in an animal, wherein the composition and the animal feed comprise one or more polypeptides having muramidase activity and one or more antibiotics as defined above.

In the sixth aspect, the invention further relates to use of one or more polypeptides having muramidase activity in reducing usage amount of an antibiotic in an animal.

Particularly, the invention relates to use of a composition or an animal feed in improving Body Weight (BW), Body Weight Gain (BWG), Feed Conversion Ratio (FCR) and European Production Efficiency Factor (EPEF) of an animal, wherein the composition and the animal feed comprise one or more polypeptides having muramidase activity and one or more antibiotics as defined above.

In the present invention, the improvement means increase of BW, BWG or EPEF, or decrease of FCR, compared to the same feed but excluding the polypeptide having muramidase activity or the antibiotics or both.

In the present invention, BW, BWG or EPEF may be increased by at least 1%, such as by at least 1.5%, at least 2.0%, at least 2.5%, at least 3%, at least 3.5%, at least 4% or at least 5%.

In the present invention, FCR may be decreased by at least 1%, such as by at least 1.5%, at least 2.0%, at least 2.5%, at least 3%, at least 3.5%, at least 4% or at least 5%.

In the present invention, the usage amount of an antibiotic in an animal are reduced by at least 10%, such as by at least 20%, at least 30%, at least 40%, at least 50%, at least 60%, at least 70%, at least 80%, at least 90% or at least 100% compared to the negative control wherein the polypeptide having muramidase activity is not present.

In the present invention, the polypeptide having muramidase activity may be dosed at a level of 10 mg to 1000 mg enzyme protein per kg animal feed, such as 50 mg to 900 mg, 100 mg to 800 mg, 200 mg to 700 mg, 300 mg to 600 mg, or 400 mg to 500 m per kg animal feed, or any combination of these intervals.

In the present invention, the antibiotics may be dosed at a level of 0.1 mg to 100 mg per kg animal feed, such as 0.5 mg to 90 mg, 1 mg to 80 mg, 5 mg to 70 mg, 10 mg to 60 mg, 15 mg to 50 mg or 20 mg to 40 mg per kg animal feed, or any combination of these intervals.

In the present invention, the animal is a mono-gastric animal, e.g. pigs or swine (including, but not limited to, piglets, growing pigs, and sows); poultry (including but not limited to poultry, turkey, duck, quail, guinea fowl, goose, pigeon, squab, chicken, broiler, layer, pullet and chick); fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish); and crustaceans (including but not limited to shrimps and prawns). In a more preferred embodiment, the animal is selected from the group consisting of swine, poultry, crustaceans and fish. In an even more preferred embodiment, the animal is selected from the group consisting of swine, piglet, growing pig, sow, chicken, broiler, layer, pullet and chick.

The present invention will be further illustrated by the following examples.

### EXAMPLES

### Example 1: Determination of Muramidase Activity

The activity of Muramidase was determined by measuring the decrease (drop) in absorbance/optical density of a solution of suspended Micrococcus lysodeikticus ATTC No. 4698 (Sigma-Aldrich M3770) measured in a microplate reader (Tecan Infinite M200) at 450 nm.

### Preparation of Micrococcus Lysodeikticus Substrate

Before use the cells were suspended in deionized water to a concentration of 10 mg cells/mL and the absorbance/optical density (OD) at 450 nm was measured. The cell suspension was then adjusted so that the cell concentration in the turbidity assay (180 μL buffer+20 μL sample+20 μL substrate) equaled an OD450=1.0. The adjusted cell suspension was then stored at ambient temperature before use. Suspended cells were used within 3 hours.

### Preparation of Citric Acid—Phosphate Buffer pH 4

61.45 mL 0.1 M citric acid was mixed with 38.55 mL 0.2 M disodium hydrogen phosphate, and the pH was adjusted with hydrochloric acid or sodium hydroxide to pH 4.

### Measurement of Muramidase Antimicrobial Activity in the Turbidity Assay

The Muramidase sample to be measured was diluted to a concentration of 50 mg enzyme protein/L in deionized water, and kept on ice until use. In a 96 well microtiter plate (Nunc) 180 μL citric acid—phosphate buffer pH 4 and 20 μL of the diluted Muramidase sample was added and kept cold (5° C.). To start the activity measurement 20 μL of the substrate (Micrococcus lysodeikticus) was added to each well, and kinetic measurement of absorbance at 450 nm was initiated for 1 hour at 37° C. in a microplate reader. The measured absorbance at 450 nm was monitored for each well and over time a drop in absorbance was seen if the Muramidase has Muramidase activity.

Following incubation, the Muramidase activity against Micrococcus lysodeikticus was determined as Δ absorbance at 450 nm (start value−end value) of each well after 1 hour. Significance was calculated using Dunnett's with control test p level 0.05 in JMP® version 12.1.0 statistical software package from SAS Institute Inc.

### Example 2: In Vivo Broiler Trial 1

### Place and Animals

The trial was performed at Poultrz section, INTA-EEA Pergamino, Buenos Aires, Argentina from Aug. 15, 2017 to Oct. 3, 2017. Day-old male broiler chickens (“Cobb-500”), were supplied by a commercial hatchery (Granja Tres Arroyos, Tres Arroyos 378, Buenos Aires, Argentina).

### Housing

Birds were housed in a conventional barn (netting open sides with curtains) into floor pens with shaving wood as litter. Gas heaters, fans and foggers were used to control the barn temperature.

All birds were weighed, separated in categories of 1 g and distributed to form homogenous lots. Birds of extreme weights were excluded.

Feed and waer were providing ad-libitum.

### Experiental Design

A randomized complete block design was used (factorial arrange, 2 Enramycin levels×2 Muramidase levels), totalizing 4 treatments with 14 reps each (252 birds per treatment, Table 3).

TABLE 3
Treatments

| Treatment | Enramycin | Muramidase |
|---|---|---|
| 1. Negative control | 0 | 0 |
| 2. Positive control | 10 ppm | 0 |
| 3. Negative control + Muramidase | 0 | 392 ppm |
| 4. Positive Control + Muramidase | 10 ppm | 392 ppm |

*Activity of muramidase: 63800 LSU(F)/g

### Experimental Diets

The experimental diets (Starter, Growing, Finisher and last week) were based on corn, soybean meal as main ingredients (Table 3). Phytase (RONOZYME® HiPhos GT, dose 1000 FYT/kg of feed, 100 g/tn of feed) was used in all the treatments. Rovimix broilders from DSM was used as vitamin-mineral premix, and maduramycin as coccidiostat. The composition of the feed was listed as Table 4

TABLE 4
Composition and nutrient contents of the basal experimental diets

| | Starter | Growing | Finisher | Last week |
|---|---|---|---|---|
| Ingredients (g/kg) | (1-14 d) | (15-28 d) | (29-42 d) | (43-49 d) |
| Corn | 59.227 | 64.413 | 67.427 | 70.500 |
| Soy oil | 0.187 | 0.630 | 0.452 | 0.367 |
| SBM (43.2%) | 35.616 | 30.521 | 28.483 | 25.526 |
| Oyster Shell | 0.705 | 0.709 | 0.712 | 0.715 |
| M&B meal | 2.884 | 2.491 | 1.846 | 1.912 |
| Coccidiostat | 0.050 | 0.050 | 0.050 | 0.000 |
| Vit-Min premix* | 0.200 | 0.200 | 0.150 | 0.150 |
| Salt | 0.466 | 0.399 | 0.414 | 0.387 |
| L-lysine HCl 78.8% | 0.198 | 0.181 | 0.123 | 0.139 |
| DL-Methionine 99% | 0.312 | 0.262 | 0.220 | 0.201 |
| L-Threonine 98% | 0.095 | 0.083 | 0.064 | 0.063 |
| Choline Cl 60% | 0.050 | 0.050 | 0.050 | 0.030 |
| Phytase** | 0.010 | 0.010 | 0.010 | 0.010 |
| Nutrients (%) | | | | |
| Curde protein | 22.00 | 20.00 | 19.00 | 18.00 |
| AME (kcal/kg) | 3.008 | 3.084 | 3.097 | 3.118 |
| TME (kcal/kg) | 3.258 | 3.336 | 3.349# | 3.372# |

*Vit-Min premix Rovimix ® Broilers and
**Ronozyme Hiphos GT from DSM Nutritional Products;
AME: Apparent metabolizable energy;
TME: true metabolizable energy.
#2% less energy than the recommendation.

### Challenge

Management tools to generate a challenge which affects the broiler performance and allows to demonstrate the beneficial effects of an antibiotic or alternatives:
Reused litter (two breeding) maintaining high moisture through the addition of 500 mL of water (every other day) up to 21 days of age
High density, 12 birds/m²
Removal of the top layer of the litter only at 28 and 42 days of age.

### Experimental Parameters and Analyses

Individual body weight and pen feed intake were determined weekly.

Based on this information feed conversion and weight gain were calculated considering each phase and the complete cycle.

The live weight/feed conversion ratio was used. This is a simplification to avoid the impact of mortality when a low number of birds per pen is used (18 in this experiment).

Uniformity (% of birds included in a range of weights of μ10% of the pen mean body weight) was determined in each pen at 48 days of age.

Every 30 minutes temperature and relative humidity were registered using a data logger.

### Results and Discussion

### Body Weight

With the Enramycin inclusion, an increment in body weight was observed (Table 5), being this difference significant from day 21 (p≤30.05). With the inclusion of muramidase, from day 21 of age, an increment in body weight was also observed (day 21, p≤0.05 and day 42, p≤10). Compared to muramidase alone, the combination of muramidase and Enramycin further increased the weight gain.

TABLE 5
body weight (g)

| | Age (days) | | | | | | |
|---|---|---|---|---|---|---|---|
| Treatments | 7 | 14 | 21 | 28 | 35 | 42 | 48 |
| 1. Negative control | 159 | 418 | 860 | 1394 | 2035 | 2617 | 3175 |
| 2. Positive control | 160 | 418 | 866 | 1402 | 2097 | 2752 | 3305 |
| 3. Negative control + | 159 | 413 | 864 | 1393 | 2057 | 2677 | 3212 |
| Muramidase | | | | | | | |
| 4. Positive Control + | 161 | 421 | 883 | 1425 | 2099 | 2762 | 3354 |
| Muramidase | | | | | | | |

### Feed Conversion Ratio

With the Enramycin inclusion, the feed conversion was improved (Table 6), being the difference significant from day 35 (p≤0.05). With the inclusion of muramidase improvements in feed conversion were observed along all the experiment, being the differences significant at day 7 and from day 21 onwards (p≤30.05). Compared to Enramycin alone, inclusion of additional muramidase further decreased feed conversion, even more than Enramycin alone at all the periods.

TABLE 6
Feed conversion ratio

| | Age (days) | | | | | | |
|---|---|---|---|---|---|---|---|
| Treatments | 7 | 14 | 21 | 28 | 35 | 42 | 48 |
| 1. Negative control | 0.842 | 1.160 | 1.358 | 1.512 | 1.647 | 1.812 | 1.941 |
| 2. Positive control | 0.816 | 1.163 | 1.348 | 1.513 | 1.615 | 1.761 | 1.885 |
| 3. Negative control + | 0.802 | 1.149 | 1.332 | 1.501 | 1.621 | 1.767 | 1.893 |
| Muramidase | | | | | | | |
| 4. Positive Control + | 0.800 | 1.139 | 1.335 | 1.494 | 1.611 | 1.750 | 1.854 |
| Muramidase | | | | | | | |

### Body Weight/Feed Conversion Ratio

With the Enramycin inclusion, the body weight/feed conversion ratio was improved (Table 7), being the differences significant from day 35 onwards (p≤0.05) and a tendency was observed at day 21 (p≤0.10). With the muramidase inclusion, an improvement in this parameter was observed, even more than Enramycin alone. This difference was significant at 21, 42 and 48 days (p≤0.05) and at 7 and 28 days (p≤0.10).

TABLE 7
Body weight/Feed conversion ratio

| | Age (days) | | | | | | |
|---|---|---|---|---|---|---|---|
| Treatments | 7 | 14 | 21 | 28 | 35 | 42 | 48 |
| 1. Negative control | 189 | 361 | 633 | 922 | 1236 | 1448 | 1641 |
| 2. Positive control | 196 | 360 | 642 | 927 | 1299 | 1565 | 1755 |
| 3. Negative control + | 198 | 360 | 649 | 928 | 1270 | 1518 | 1698 |
| Muramidase | | | | | | | |
| 4. Positive Control + | 202 | 370 | 662 | 954 | 1304 | 1580 | 1810 |
| Muramidase | | | | | | | |

### Other Zootechnical Parameters

With the inclusion of Enramycin, a reduction equivalent to 1.1 days to reach the slaughtering weight (2800 g) was observed (p≤0.05, Table 8). With Muramidase, a reduction of 0.3 days to reach 2800 g of body weight was observed At 48 days of age, no differences in uniformity between treatments was observed (p>0.05).

TABLE 8
Slaughtering age and uniformity at 48 days of age

| | Age to 2800 g* | Uniformity |
|---|---|---|
| Treatments | days | % |
| 1. Negative control | 43.94 | 87.03 |
| 2. Positive control | 42.61 | 88.09 |
| 3. Negative control + Muramidase | 43.40 | 87.40 |
| 4. Positive Control + Muramidase | 42.38 | 89.14 |

### Conclusion

The results obtained in the study showed that the inclusion of microbial muramidase alone or together with antibiotic (Enramycin) was effective in improving growth performance of broilder chickens. Accordingly, it becomes possible to reduce the usage amount of antibiotic for broilder chickens.

### Example 3: In Vivo Broiler Trial 2

### Outline of the Study

The feeding trial was conducted with a male flock of 2400 Cobb 400Y chicks which for a period of 35 days. The chicks were procured from a commercial hatchery and were raised throughout the study period on litter in pens. Distribution of the chicks into 6 treatment groups and into pens (6.5×4 feet) within a group was done following a completely randomized block design. Each treatment group consisted of 16 replicate pens and there were 25 chicks in each pen (n=400 per treatment and total number of chicks will be 2400).

The treatment groups were as follows:

TABLE 9
Treatments

| | | Bacitracin | |
|---|---|---|---|
| | | Methylene | |
| Treatment | | Disalicylate | Muramidase |
| T1 | Negative control (NC) | 0 | 0 |
| T2 | Positive control (PC) | 50 ppm | 0 |
| T3 | NC + Muramidase (NB25) | 0 | 450 ppm |
| | | | (25,000 LSU(F)/kg) |
| T4 | PC + Muramidase (PB25) | 50 ppm | 450 ppm |
| | | | (25,000 LSU(F)/kg) |
| T5 | NC + Muramidase (NB45) | 0 | 810 ppm |
| | | | (45,000 LSU(F)/kg) |
| T6 | PC + Muramidase (PB45) | 50 ppm | 810 ppm |
| | | | (45,000 LSU(F)/kg) |

### Feed

All feed was given in mash form. Three phase feeding were practised: Pre-starter (1-14 d/500 g/bird), Starter (15-28 d/1500 g/bird) and Finisher (29-35 d/feed till liquidation). Raw materials of same lot were used for preparation of the diets and diets of all stages were prepared afresh just prior to the start of the respective feeding stage. All diets were formulated following the ideal protein ratio using standardized ileal digestible amino acid requirement of the birds (Table 10).

TABLE 10
Composition and nutrient contents of the basal experimental diets

| | Pre starter | Starter | Finisher |
|---|---|---|---|
| | (1-14 d) | (15-28 d) | (29-42 d) |
| Maize | 565 | 609.6 | 661.1 |
| oil-veg | 26.1 | 33 | 31.360 |
| Soya DOC 45% | 336.1 | 289.4 | 243.9 |
| Meat cum bone meal | 40 | 40 | 40 |
| salt | 4.666 | 4.667 | 4.665 |
| Sodium bi-carbonate | 1.000 | 1.000 | 1.000 |
| Dicalcium phosphate | 10.440 | 8.51 | 5.94 |
| LSP-powder | 7.351 | 5.338 | 4.349 |
| DL-Methionine | 3.259 | 2.671 | 2.199 |
| L-Lysine HCL | 2.187 | 2.034 | 2.033 |
| L-Threonine | 0.659 | 0.386 | 0.216 |
| Choline Chloride, 75% | 1.000 | 1.000 | 1.000 |
| Vitamin Premix | 0.500 | 0.500 | 0.500 |
| Trace Mineral Mixture | 1.200 | 1.200 | 1.200 |
| Coccidiostat (Bobenidine 10%) | 0.500 | 0.500 | 0.500 |
| Toxin Binder | 1.000 | 1.000 | 1.000 |
| Total | 1001 | 1001 | 1001 |

Nutrient Name

| | | | |
|---|---|---|---|
| M.E (Kcal/kg) | 3000 | 3100 | 3150 |
| Protein (%) | 22.59 | 20.77 | 19.09 |
| Dig. Lysine (%) | 1.25 | 1.12 | 1.01 |
| Dig.Methionine(%) | 0.64 | 0.56 | 0.49 |
| Dig TSAA (%) | 0.929 | 0.832 | 0.750 |
| Calcium (%) | 0.880 | 0.760 | 0.660 |
| Available Phosphorus (%) | 0.420 | 0.380 | 0.330 |
| Sodium (%) | 0.200 | 0.200 | 0.200 |
| Dig. Threnine (%) | 0.829 | 0.742 | 0.669 |
| dig. Leucine (%) | 1.734 | 1.630 | 1.536 |
| dig. Iso-leucine (%) | 0.842 | 0.764 | 0.690 |
| dig. Valine | 0.967 | 0.865 | 0.787 |

### Vaccination

The birds were vaccinated according to the standard vaccination procedure through Intra Ocular route against Newcastle disease (5 d and 12 d) and infectious bursal disease (20 d).

### Parameters to be Measured

1. Performance traits (Body weights, Average daily body weightin, Average daily feed intake and Feed Conversion Ratio) will be calculated pen wise.

2. European Production Efficiency Factor (EPEF) calculated by replicate.

EPEF=ADG (g)/FCR*10)×(100−% mortality)

### Statistical Analysis

All data were analysed statistically according to a 2×3 factorial design using the levels of supplemental BMD (0 and 50 ppm) and that of muramidase (0, 450 and 810 ppm) as the main factors. The main effects and interactions were determined. Another component, viz., the effect of house, was also be added to the statistical model to see if distribution of the experimental flocks into the two separate houses elicit any effect on performance of the birds. The results were expressed as mean and pooled standard error of mean and whenever there is any significant variation (P<0.05), mean separation was done following Tukey's B test.

### Results

Performance in terms of body weight gain (BWG), feed intake (FI) and feed conversation ratio (FCR) was recorded at weekly interval from day 1 to day 35. As shown in Table 11, supplementation of muramidase even at the lower concentration (450 ppm) in NC, BWG increased significantly compared the unsupplemented group. The BWG increased progressively with increase dose of Muramidase from 450 to 810 mg/kg when the product was supplemented to PC. The feed required to produce a unit of live body weight gain was also reduced with inclusion of muramidase at 450 mg/kg diet when the product of supplemented to either PC or NC. However, the feed efficiency was better when the muramidase was supplemented to PC compared to those fed NC.

TABLE 11
Effect of supplementing Muramidase on performance of broilers
fed diets with and without antibiotic growth promoter

| | | 35th day | | | |
|---|---|---|---|---|---|
| Diets | Muramidase | BWG, g | FI, g | FI/BWG | EPEF |
| NC | 0 | 2090^{c} | 3284 | 1.572^{ab} | 372.3 |
| PC | 0 | 2074^{c} | 3275 | 1.579^{a} | 374.5 |
| NC | 450 | 2141^{b} | 3316 | 1.549^{c} | 387.9^{b} |
| PC | 450 | 2153^{b} | 3297 | 1.531^{d} | 398.7^{a} |
| NC | 810 | 2114^{bc} | 3301 | 1.562^{b} | 384.9^{bc} |
| PC | 810 | 2206^{a} | 3368 | 1.527^{d} | 406.5^{a} |
| SEM | | 7.791 | 10.26 | 0.003 | 1.957 |
| Diets | NC | 2115 | 3300 | 1.561 | 381.7^{b} |
| | PC | 2144 | 3313 | 1.546 | 393.2^{a} |
| SEM | | 9.274 | 14.201 | 0.002 | 2.186 |
| Murami- | 0 | 2082^{b} | 3279 | 1.575^{a} | 373.4^{b} |
| dase | 450 | 2147^{a} | 3306 | 1.540^{b} | 393.3^{a} |
| | 810 | 2160^{a} | 3335 | 1.544^{b} | 395.7^{a} |
| SEM | | 11.359 | 17.393 | 0.003 | 2.677 |
| P values | | | | | |
| Diets | | 0.026 | 0.507 | 0.001 | 0.001 |
| Bond | | 0.001 | 0.088 | 0.001 | 0.001 |
| Interaction | | 0.003 | 0.161 | 0.001 | 0.042 |

BWG: body weight gain;
FI: feed intake;
NC: negative control;
PC: positive control;
P: probability;
SEM: standard error mean
^{abc}means having common superscript in a column do not vary significantly (P, 0.05)

The interaction between supplementation of the antibiotic and muramidase significantly influenced the EPEF in broiler chicken from day 1 to 35d of age (Table 11). Supplementation of muramidase to both NC and PC at the lowest concentration tested (450 mg/kg) significantly improved the EPEF compared to the unsupplemented control group. The EPEF was significantly higher with muramidase supplementation to PC compared to those fed NC with muramidase inclusion.

### Conclusions

Based on the performance data, it could be concluded that supplementation of muramidase alone or in combination with an antibiotic significantly improved the body weight gain, feed efficiency and European performance efficiency factor (EPEF) in commercial broilers reared under challenge rearing condition (meat cum bone meal in diet and built up litter) compared to un supplemented group.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A composition comprising one or more polypeptides having muramidase activity and one or more antibiotics.

2. The composition of claim 1, wherein the polypeptide having muramidase activity is a fungal GH24 muramidase or GH25 muramidase.

3. The composition of claim 1, wherein the antibiotic is any one of Bacitracin, Bambermycin, Carbadox, Enramycin, Enduracidin, Laidlomycin, Lasalocid, Lincomycin, Monensin, Neomycin, Penicillin, Roxarsone, Roxarsone, Salinomycin, Tylosin, and Virginiamycin.

4. The composition of claim 1, wherein the polypeptide having muramidase activity is selected from the group consisting of:
(a) a polypeptide having at least 80%, e.g., at least 85%, at least 90%, at least 95%, or 100% sequence identity to SEQ ID NO: 1;
(b) a polypeptide having at least 80%, e.g., at least 85%, at least 90%, at least 95%, or 100% sequence identity to SEQ ID NO: 2;
(c) a polypeptide having at least 80%, e.g., at least 85%, at least 90%, at least 95%, or 100% sequence identity to SEQ ID NO: 3;
(d) a polypeptide having at least 80%, e.g., at least 85%, at least 90%, at least 95%, or 100% sequence identity to SEQ ID NO: 4;
(e) a polypeptide having at least 80%, e.g., at least 85%, at least 90%, at least 95%, or 100% sequence identity to SEQ ID NO: 5;
(f) a polypeptide having at least 80%, e.g., at least 85%, at least 90%, at least 95%, or 100% sequence identity to SEQ ID NO: 6;
(g) a polypeptide having at least 80%, e.g., at least 85%, at least 90%, at least 95%, or 100% sequence identity to SEQ ID NO: 7;
(h) a polypeptide having at least 80%, e.g., at least 85%, at least 90%, at least 95%, or 100% sequence identity to SEQ ID NO: 8;
(i) a polypeptide having at least 80%, e.g., at least 85%, at least 90%, at least 95%, or 100% sequence identity to SEQ ID NO: 9;
(j) a polypeptide having at least 80%, e.g., at least 85%, at least 90%, at least 95%, or 100% sequence identity to SEQ ID NO: 10;
(k) a polypeptide having at least 80%, e.g., at least 85%, at least 90%, at least 95%, or 100% sequence identity to SEQ ID NO: 11;
(l) a polypeptide having at least 80%, e.g., at least 85%, at least 90%, at least 95%, or 100% sequence identity to SEQ ID NO: 12;
(m) a polypeptide having at least 80%, e.g., at least 85%, at least 90%, at least 95%, or 100% sequence identity to SEQ ID NO: 13;
(n) a polypeptide having at least 80%, e.g., at least 85%, at least 90%, at least 95%, or 100% sequence identity to SEQ ID NO: 14;
(o) a polypeptide having at least 80%, e.g., at least 85%, at least 90%, at least 95%, or 100% sequence identity to SEQ ID NO: 15;
(p) a polypeptide having at least 80%, e.g., at least 85%, at least 90%, at least 95%, or 100% sequence identity to SEQ ID NO: 16;
(q) a polypeptide having at least 80%, e.g., at least 85%, at least 90%, at least 95%, or 100% sequence identity to SEQ ID NO: 17;
(r) a polypeptide having at least 80%, e.g., at least 85%, at least 90%, at least 95%, or 100% sequence identity to SEQ ID NO: 18;
(s) a polypeptide having at least 80%, e.g., at least 85%, at least 90%, at least 95%, or 100% sequence identity to SEQ ID NO: 19;
(t) a polypeptide having at least 80%, e.g., at least 85%, at least 90%, at least 95%, or 100% sequence identity to SEQ ID NO: 20;
(u) a polypeptide having at least 80%, e.g., at least 85%, at least 90%, at least 95%, or 100% sequence identity to SEQ ID NO: 21;
(v) a polypeptide having at least 80%, e.g., at least 85%, at least 90%, at least 95%, or 100% sequence identity to SEQ ID NO: 22;
(w) a polypeptide having at least 80%, e.g., at least 85%, at least 90%, at least 95%, or 100% sequence identity to SEQ ID NO: 23;
(x) a polypeptide having at least 80%, e.g., at least 85%, at least 90%, at least 95%, or 100% sequence identity to SEQ ID NO: 24;
(y) a polypeptide having at least 80%, e.g., at least 85%, at least 90%, at least 95%, or 100% sequence identity to SEQ ID NO: 25;
(z) a polypeptide having at least 80%, e.g., at least 85%, at least 90%, at least 95%, or 100% sequence identity to SEQ ID NO: 26;
(aa) a polypeptide having at least 80%, e.g., at least 85%, at least 90%, at least 95%, or 100% sequence identity to SEQ ID NO: 27;
(ab) a polypeptide having at least 80%, e.g., at least 85%, at least 90%, at least 95%, or 100% sequence identity to SEQ ID NO: 28;
(ac) a polypeptide having at least 80%, e.g., at least 85%, at least 90%, at least 95%, or 100% sequence identity to SEQ ID NO: 29;
(ad) a polypeptide having at least 80%, e.g., at least 85%, at least 90%, at least 95%, or 100% sequence identity to SEQ ID NO: 30;
(ae) a polypeptide having at least 80%, e.g., at least 85%, at least 90%, at least 95%, or 100% sequence identity to SEQ ID NO: 31;
(af) a polypeptide having at least 80%, e.g., at least 85%, at least 90%, at least 95%, or 100% sequence identity to SEQ ID NO: 32;
(ag) a polypeptide having at least 80%, e.g., at least 85%, at least 90%, at least 95%, or 100% sequence identity to SEQ ID NO: 33;
(ah) a polypeptide having at least 80%, e.g., at least 85%, at least 90%, at least 95%, or 100% sequence identity to SEQ ID NO: 34;
(al) a polypeptide having at least 80%, e.g., at least 85%, at least 90%, at least 95%, or 100% sequence identity to SEQ ID NO: 35;
(aj) a polypeptide having at least 80%, e.g., at least 85%, at least 90%, at least 95%, or 100% sequence identity to SEQ ID NO: 36;
(ak) a polypeptide having at least 80%, e.g., at least 85%, at least 90%, at least 95%, or 100% sequence identity to SEQ ID NO: 37;
(al) a polypeptide having at least 80%, e.g., at least 85%, at least 90%, at least 95%, or 100% sequence identity to SEQ ID NO: 38;
(am) a polypeptide having at least 80%, e.g., at least 85%, at least 90%, at least 95%, or 100% sequence identity to SEQ ID NO: 39;
(an) a polypeptide having at least 80%, e.g., at least 85%, at least 90%, at least 95%, or 100% sequence identity to SEQ ID NO: 40;
(ao) a polypeptide having at least 80%, e.g., at least 85%, at least 90%, at least 95%, or 100% sequence identity to SEQ ID NO: 41;
(ap) a polypeptide having at least 80%, e.g., at least 85%, at least 90%, at least 95%, or 100% sequence identity to SEQ ID NO: 42;
(aq) a polypeptide having at least 80%, e.g., at least 85%, at least 90%, at least 95%, or 100% sequence identity to SEQ ID NO: 43;
(ar) a polypeptide having at least 80%, e.g., at least 85%, at least 90%, at least 95%, or 100% sequence identity to SEQ ID NO: 44;
(as) a polypeptide having at least 80%, e.g., at least 85%, at least 90%, at least 95%, or 100% sequence identity to SEQ ID NO: 45;
(at) a polypeptide having at least 80%, e.g., at least 85%, at least 90%, at least 95%, or 100% sequence identity to SEQ ID NO: 46;
(au) a polypeptide having at least 80%, e.g., at least 85%, at least 90%, at least 95%, or 100% sequence identity to SEQ ID NO: 47;
(av) a polypeptide having at least 80%, e.g., at least 85%, at least 90%, at least 95%, or 100% sequence identity to SEQ ID NO: 48;
(aw) a polypeptide having at least 80%, e.g., at least 85%, at least 90%, at least 95%, or 100% sequence identity to SEQ ID NO: 49;
(ax) a polypeptide having at least 80%, e.g., at least 85%, at least 90%, at least 95%, or 100% sequence identity to SEQ ID NO: 50;
(ay) a polypeptide having at least 80%, e.g., at least 85%, at least 90%, at least 95%, or 100% sequence identity to SEQ ID NO: 51;
(az) a polypeptide having at least 80%, e.g., at least 85%, at least 90%, at least 95%, or 100% sequence identity to SEQ ID NO: 52;
(ba) a polypeptide having at least 80%, e.g., at least 85%, at least 90%, at least 95%, or 100% sequence identity to SEQ ID NO: 53;
(bb) a polypeptide having at least 80%, e.g., at least 85%, at least 90%, at least 95%, or 100% sequence identity to SEQ ID NO: 54;
(bc) a polypeptide having at least 80%, e.g., at least 85%, at least 90%, at least 95%, or 100% sequence identity to SEQ ID NO: 55;
(bd) a polypeptide having at least 80%, e.g., at least 85%, at least 90%, at least 95%, or 100% sequence identity to SEQ ID NO: 56;
(be) a polypeptide having at least 80%, e.g., at least 85%, at least 90%, at least 95%, or 100% sequence identity to SEQ ID NO: 57;
(bf) a polypeptide having at least 80%, e.g., at least 85%, at least 90%, at least 95%, or 100% sequence identity to SEQ ID NO: 58;
(bg) a polypeptide having at least 80%, e.g., at least 85%, at least 90%, at least 95%, or 100% sequence identity to SEQ ID NO: 59;
(bh) a polypeptide having at least 80%, e.g., at least 85%, at least 90%, at least 95%, or 100% sequence identity to SEQ ID NO: 60;
(bi) a polypeptide having at least 80%, e.g., at least 85%, at least 90%, at least 95%, or 100% sequence identity to SEQ ID NO: 61;
(bj) a polypeptide having at least 80%, e.g., at least 85%, at least 90%, at least 95%, or 100% sequence identity to SEQ ID NO: 62;
(bk) a polypeptide having at least 80%, e.g., at least 85%, at least 90%, at least 95%, or 100% sequence identity to SEQ ID NO: 63;
(bl) a polypeptide having at least 80%, e.g., at least 85%, at least 90%, at least 95%, or 100% sequence identity to SEQ ID NO: 64;
(bm) a polypeptide having at least 80%, e.g., at least 85%, at least 90%, at least 95%, or 100% sequence identity to SEQ ID NO: 65;
(bn) a polypeptide having at least 80%, e.g., at least 85%, at least 90%, at least 95%, or 100% sequence identity to SEQ ID NO: 66;
(bo) a polypeptide having at least 80%, e.g., at least 85%, at least 90%, at least 95%, or 100% sequence identity to SEQ ID NO: 67;
(bp) a polypeptide having at least 80%, e.g., at least 85%, at least 90%, at least 95%, or 100% sequence identity to SEQ ID NO: 68;
(bq) a polypeptide having at least 80%, e.g., at least 85%, at least 90%, at least 95%, or 100% sequence identity to SEQ ID NO: 69;
(br) a polypeptide having at least 80%, e.g., at least 85%, at least 90%, at least 95%, or 100% sequence identity to SEQ ID NO: 70;
(bs) a polypeptide having at least 80%, e.g., at least 85%, at least 90%, at least 95%, or 100% sequence identity to SEQ ID NO: 71;
(bt) a variant of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70 or SEQ ID NO: 71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions;
(bu) a polypeptide comprising the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), (bs) or (bt) and a N-terminal and/or C-terminal extension of between 1 and 10 amino acids; and
(bv) a fragment of a polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), (bs) or (bt) having muramidase activity and having at least 90% of the length of the mature polypeptide.

5. The composition of claim 1, wherein the antibiotic is Enramycin or Bacitracin.

6. The composition of claim 1, which comprises the muramidase of SEQ ID NO: 1 and Enramycin or Bacitracin.

7. An animal feed comprising an animal feed additive, one or more protein sources and one or more energy sources, wherein the animal feed further comprises one or more polypeptides having muramidase activity and one or more antibiotics.

8. The animal feed of claim 7, wherein the protein source is selected from the group consisting of soybean, wild soybean, beans, lupin, tepary bean, scarlet runner bean, slimjim bean, lima bean, French bean, Broad bean (fava bean), chickpea, lentil, peanut, Spanish peanut, canola, sunflower seed, cotton seed, rapeseed (oilseed rape) or pea or in a processed form such as soybean meal, full fat soy bean meal, soy protein concentrate (SPC), fermented soybean meal (FSBM), sunflower meal, cotton seed meal, rapeseed meal, fish meal, bone meal, feather meal, whey or any combination thereof.

9. The animal feed of claim 7, wherein the energy source is selected from the group consisting of maize, corn, sorghum, barley, wheat, oats, rice, triticale, rye, beet, sugar beet, spinach, potato, cassava, quinoa, cabbage, switchgrass, millet, pearl millet, foxtail millet or in a processed form such as milled corn, milled maize, potato starch, cassava starch, milled sorghum, milled switchgrass, milled millet, milled foxtail millet, milled pearl millet, or any combination thereof.

10. A method of improving growth performance in an animal comprising administering to the animal the composition according to claim 1 or the animal feed.

11. The method of claim 10, wherein the composition or the animal feed is dosed so that the antibiotic is at a level of 0.1 mg to 100 mg per kg animal feed, such as 0.5 mg to 90 mg, 1 mg to 80 mg, 5 mg to 70 mg, 10 mg to 60 mg, 15 mg to 50 mg or 20 mg to 40 mg per kg animal feed, or any combination of these intervals.

12. The method of claim 10, wherein the composition or the animal feed is dosed so that the polypeptide having muramidase activity is at a level of 10 mg to 1000 mg enzyme protein per kg animal feed, such as 50 mg to 900 mg, 100 mg to 800 mg, 200 mg to 700 mg, 300 mg to 600 mg, or 400 mg to 500 m per kg animal feed, or any combination of these intervals.

13. The method of claim 10, wherein the animal is a mono-gastric animal, e.g. pigs or swine (including, but not limited to, piglets, growing pigs, and sows); poultry (including but not limited to poultry, turkey, duck, quail, guinea fowl, goose, pigeon, squab, chicken, broiler, layer, pullet and chick); pet animals such as cats and dogs, fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish); and crustaceans (including but not limited to shrimps and prawns).

14. Use of the composition according to claim 1 or the animal feed in improving growth performance in an animal.

15. A method for reducing usage amount of an antibiotic in an animal comprising administering to the animal one or more polypeptides having muramidase activity.

16. Use of one or more polypeptides having muramidase activity in reducing usage amount of an antibiotic in an animal.


SEQUENCE LISTING (71 sequences)

SEQ ID NO: 1 - protein, 208 aa, Acremonium alcalophilum
RIPGFDISGWQPTTDFARAYANGDRFVYIKATEGTTFKSSAFSRQYTGATQNGFIRGAYHFAQPAASSGAAQARYFASNGGGWSKDGITLPGALDIEYNPNGATCYGLSQSAMVNWIEDFVTTYHGITSRWPVIYTTTDWWTQCTGNSNRFANRCPLWIARYASSVGTLPNGWGFYTFWQYNDKYPQGGDSNWFNGDASRLRALANGD

SEQ ID NO: 2 - protein, 213 aa, Acremonium alcalophilum
SPIRRRIPGFDISGWQPTTDFARAYANGDRFVYIKATEGTTFKSSAFSRQYTGATQNGFIRGAYHFAQPAASSGAAQARYFASNGGGWSKDGITLPGALDIEYNPNGATCYGLSQSAMVNWIEDFVTTYHGITSRWPVIYTTTDWWTQCTGNSNRFANRCPLWIARYASSVGTLPNGWGFYTFWQYNDKYPQGGDSNWFNGDASRLRALANGD

SEQ ID NO: 3 - protein, 218 aa, Aspergillus fumigatus
LPSQPEARATTVQGFDISNHQKSVNFEAAKKDGAQFVMIKATEGTTYKDTVFNSHYTGATKAGLLRGGYHFARPDKSTGSTQAKFFLKNGGGWSDDNRTLPGMLDIEYNPYGATCYGLSHSQMVAWIHDFVNEYHHATSRWPMIYTTADWWNRCTGNAKGFGDKCPLVLAAYSSSPPKTIPGDWKTWTIWQNSDKYKHGGDSDKFNGPMTQLRKLASG

SEQ ID NO: 4 - protein, 208 aa, Trichoderma reesei
TVPGFDISHYQATVDFAKAYADGARFVIIKATEGTTYTDPSFSDHYTKATNAGFIRGGYHFAQPASSSGAAQANYFLKHGGGWSADGITLPGMLDLEYAPSGDSCYGLSASAMVSWINDFVNTYHAATTQYPLIYTSTSWWQLCTGNNGSFGSKSPLVIARYASSVGALPNGWSVYTIWQNSDASPWGGDNDIFNGNLAQLQKIARGS

SEQ ID NO: 5 - protein, 215 aa, Trametes cinnabarina
SPTPEKRANPKGIDVSAYQPNINWSTVKANGISFAYIKATEGTTYTNPDFSSQYTGATNAGLIRGGYHFAHPDSSSGATQAKYFLAHGGGWTSDGITLPGALDIEYNPSGAECYGLSASAMVSWIKDFSNTYHSSTGVYPVIYTTTDWWTTCTGNSAAFASTNPLWIARYASSIGTLPAGWSYTTFWQYADSGPNPGDQDEFNGSMAGLKQLALG

SEQ ID NO: 6 - protein, 207 aa, Sporormia fimetaria
AVPGFDISHYQSSVNYAGAYSSGARFVIIKATEGTTYIDPKFSDHYIGATNAGLIRGAYHFARPAASTGAAQANYFVSHGGGWSADGITLPGMLDMEYGSTSACHGLSQSAMVTWITSFVNQYNSLTGRYPMIYTTADWWQTCTGNSAAFNTKSPLVLARYSSSAGTVPGGWPYYTIWQFNDAYAYGGDSDTFNGDLAGLKRLAKGS

SEQ ID NO: 7 - protein, 201 aa, Poronia punctata
AVQGFDISHYQSSVNFGAAKSSGAQFVIIKATEGTSYTDPSFSSHYTGATNAGLIRGGYHFAHLDSSSGAAQAKYFLAHGGGWSGDGITLPGMLDLEGSCVLSASATVSWIKDFSNTYHSSTGVYPLIYTNPSWWSSCTGNSKAFIDTNPLVLARYASSAGTPPGGWPYYTIWQYNDAYKYGGDSDVFNGDLAGLKRLAKG

SEQ ID NO: 8 - protein, 201 aa, Poronia punctata
QVQGFDISSYQPSVDFAGAYADGARFVIIKATEGTGYIDPTFSDHYVGATNAGLLRGGYHYAHLDSTSGATQAQYFLANGGGWSGDGITLPGMLDLEGDCVLSAADAVAWIKDFSDTYHASTGVYPLLYTNPSWWASCTGDSSAFIDTNPLVLAHYADAAGTPPGGWPFYSFWQYNDAYPYGGDSEVWNGDMDGLLRLASG

SEQ ID NO: 9 - protein, 203 aa, Lecanicillium sp. WMM742
VDSSSEVSVAIYKKALGQGFTRAIFRGYQEACSQGGRVDPTFVPSYKNAVAAGYKDFDAYFFPCTGKTNKCKPYAAQLAELLDTIKGQKLAIRRIWLDIETDRVCNPFDYGAQGNLAEAKKLVAAFRDAKLDWGIYTSPTQWETIFGAKTVELAKDVPLWFAKFDNVETLELKTPFGGWTKADAKQYTDQSASNKFDLNVFSA

SEQ ID NO: 10 - protein, 208 aa, Lecanicillium sp. WMM742
TVQGFDVSGYQPTVNWGAAYSSGARFVMIKATEGTGYISSSFGSQYPGATNAGFIRGGYHFALPDRSSGSAQADYFLAHGGGWSGDGITLPGMLDIEYNPYGATCYGLSQGAMVNWISDFVEHYKARTTQYPIIYTTTDWWKTCTGNSPAFGQKCPLSLARYSSSVGEIPNGWPFQTFWQNSDKYAYGGDSQIFNGAYSQLQKIARGG

SEQ ID NO: 11 - protein, 207 aa, Onygena equina
AVPGIDVSGYQGNVNWANVANAGKKFAYVKATEHTNYINPYFAQQYNGAYNQGIIRGAYHYAHPNGASGASQANYFLAHGGGWSADGKTLPGAVDLEYGPNGSTCWGISQSAMIAWIRDFSNTYRAKTGRPPVIYTSTSWWKTCTGNYGGFGNDNPLWIARYSSTVGELPAGWPFHSIWQNNDNSGVGGDGDIWNGDLAGLQRFAKG

SEQ ID NO: 12 - protein, 208 aa, Purpureocillium lilacinum
AVKGFDISHYQPNVDFAKAYADGARFVMIKATEGTTYTDPSFSSHYTGATKAGFIRGGYHFARPASSSGAAQAKYFIAHGGGWSKDGITLPGMLDMEYQSSSSACGGLSQSAMVSWINDFVNTYHAATGVYPLIYTSTSWWTQCTGNSAAFGSKCPLVVARYASSVGTLPAGWGFYTFWQYSDAAPWGGDADTFNGDITALKKIANAG

SEQ ID NO: 13 - protein, 207 aa, Trichobolus zukalii
AVPGFDISHYQPSVNYAGAYNSGARFVIIKATEGTTYTDPVFSTHYTGATKAGLIRGGYHFARPASSSGSAQADFFFKNGGGWSADGITLPGMLDMEYGSTSSCHGLSQTAMVNWISDFVNRYKTLSGRYPMIYTGYYWWVECTGNSNKFATTCPLVLARYSSSVGEIPGGWGYQTIWQFNDKYAYGGDSDSFNGSLDRLKALAKGT

SEQ ID NO: 14 - protein, 207 aa, Penicillium citrinum
LIHAVDSSSEVSVDIYKKALSEGFSRAIFRGYQEACSQGGRVDPTFLPSYKNAQTAGYKDFDAYFFPCTGSGNKCKPYDVQIGELVDAIKKNNMAIRRIWVDFEKDKTCNPFNWDPKRNIDEAKRIIGAVRKTKFDFGVYTSATQWTSIFGSKDVVLANDVPLWFAKFDNVENLDLAQPFGGWTKADGKQYTDKSASKKFDLNVFSA

SEQ ID NO: 15 - protein, 207 aa, Cladorrhinum bulbillosum
AVQGFDISHYQSSVNFQAAYNSGARFVIIKATEGTTYIDPKFSSHYTGATNAGLIRGGYHFAHPDSSTGAAQADFFLAHGGGWSGDGITLPGMLDLESVSGKATCFGLSASSMVAWIKSFSDRYHTRTGRYPMLYTNPSWWTTCTGNSNAFVNTNPLVLARYASAPGTIPGGWPYQTIWQNSDSYTYGGDSDIFNGALSGLQKLASG

SEQ ID NO: 16 - protein, 208 aa, Umbelopsis westeae
KLKGLDVSGYQPNVAWSTVKANGASFAYIKATEGTNYKNPSFAQQYNGAYNAGLIRGSYHFAQPSSSTGAAQANYFLAHGGGWSPDGKTLPGALDMEYNPHGSTCYGLSKDAMVKWIKDFSNTYHSATGRYPVIYTTTSWWTTCTGNSAAFGATNPLWIARYSSTAGNLPNGWAFYSFWQNADSGIFPGDQDIWNGDAAALSRMAKGA

SEQ ID NO: 17 - protein, 208 aa, Zygomycetes sp. XZ2655
TLPGLDVSSYQGNVNWGTVASQGAKFAYVKATEGTTYTNPYFASQYDGSYNAGLIRGAYHFAHPDSSSGATQANYFLAHGGGWSADGKTLPGALDIEYNPNGAECYGLSQLAMISWIQDFSNTYHSHTGRYPVIYTTTDWWTTCTGNSAAFGTNNPLWIARYSSSVGTLPAGWGYESFWQKASSGTFPGDQDIWNGDAAGLSRFATGK

SEQ ID NO: 18 - protein, 206 aa, Chaetomium cupreum
TVQGFDISGYQPNVNFAAAYAAGARFVIIKATEGTSYISPSFSSQYTGATNAGFIRGGYHFAHPGASSGTTQADYFIAHGGGWTPDGITLPGMLDLESESSGTCWGLSASAMVAWIKDFSDHYHSRMGVYPLLYTNPSWWEECTGNSNAFVDTNPLVLAHYSSSVGTIPGGWPYETIWQNSDSYAYGGDSDVFNGDLAGLQRLARG

SEQ ID NO: 19 - protein, 207 aa, Cordyceps cardinalis
LIHAVDSSSEVSVDVYKKALAEGFTRAIFRGYQEACSQGGRVDPTFLPSYKNAQKAGYKDFDAYFFPCTGSGNKCKPYAKQIGELVDAIEGNQLAIRRIWIDIETDKVCNAFNWGAEGNIQEAKKLIAAVRGTKRDFGIYTSATQWENIFGSRTVELAKDVPLWFAKFDNVETLELKTPFGGWTKADAKQYTDKSASKKFDLNVFSA

SEQ ID NO: 20 - protein, 216 aa, Penicillium sp. ′qii′
STIQPRASGVQGFDISSYQGTVNFAGAYGAGARFVMIKATEGTTYIDSTFSSHYDGATSAGLIRGAYHFAHPDSSSGATQAEYFLAHGGGWTNDGITLPGMLDIEYNPSGSTCYGLSASAMVSWIKDFGETYNSKTGRYPMIYSTADWWSTCTGDSTSFSSDYPLVLAQYASSISTVPGGWPYQSFWQNADSYSYGGDSDLWNGSEDSLKTFAKGS

SEQ ID NO: 21 - protein, 218 aa, Aspergillus sp. nov XZ2609
LPTKLAARYSTVQGFDVSNYQPNVDFSAAKSAGAEFVIIKATEGTDYKDTYFNSHYTGATNAGLIRGGYHFARPDKSSGTAQAEYFLAHGGGWSKDGRTLPGMLDIEYNPYGATCYGLSHSAMVSWVNEFLNEYHSKTGVYPLLYTTADWWNQCTGNAHGFGNKSPLVLASYSSESPRTVPGDWQTWTIWQNADKYKYGGDSDIFNGDLTQLKKIVEG

SEQ ID NO: 22 - protein, 204 aa, Paecilomyces sp. XZ2658
TVAGFDISNYQPSVNFAKAYADGARFATEGTTYIDPSFSSHYTGATNAGLIRGGYHFAHPGSSTGAAQATYFLAHGGGWSKDGITLPGMIDLEYNPSGATCYGLSTSAMVSWISDFVETYHSKTGVYPLIYTSTSWWNQCTGSSTAFASKCPLVVARYASSVGTLPAGWGYQTIWQNSDSSPWGGDNDIFNGSLDQLKRIANAS

SEQ ID NO: 23 - protein, 203 aa, Paecilomyces sp. XZ2658
AVQGHDVSHWQGNINWGAVKAAGVKFTYIKATESTNYIDPSFNANYVGATNTGLIRGAYHFARPGDSSGAAQANYFVSHGGGWSADGRTLPGALDLEAGCSGLSQSAMTAWIRDFSNTYHARTGRFPVIYTTTSWWKTCTGNASGFQNDHPLWIARWGPSPGELPAGYGFHTFWQYADKGPLPGDQDNFNGDEAGLARLARGS

SEQ ID NO: 24 - protein, 208 aa, Pycnidiophora cf dispera
AVSGMDISHYQGTNYNFAGAYSSGARFVIIKATEGTTYTDPQFSANYIGATNAGFIRGAYHFARPAASTGAVQASYFVSHGGGWSSDGITLPGMLDMEYGSTSTCHGLSVSAMNTWIASFVNQYKSLTGAYPMIYTTADWWKTCTGDSTAWNTKCPLVLARYSSSVGTIPGGWPYHTIWQYSDSYAYGGDSDTFNGDLAGLKRLAKGS

SEQ ID NO: 25 - protein, 210 aa, Thermomucor indicae-seudaticae
YQTGLDVSALTSTSSFSCAKNLGYDHVIARCYMEAYGNNPGGKVDPNCYSNYKNAKAAGFTSVDIYMFPCTGRSTCKSPATQVQEIVDYVGAHKMIVGTLWLDVEVDSAANNWPSTSEARSTLRAFKTALDKSGWKWGVYSSKSQWTRITGSASWVLDPSVPLWYSHYDDTLSFSDYPSHAFGGWSKPTIKQYTGDASFCSASWDKNYYG

SEQ ID NO: 26 - protein, 207 aa, Isaria farinosa
LTHAVDSSSEVSVDIYKKALGQGFTRAIFRGYQEACSLGGRVDPTFVPSYKNAVAAGYKDFDAYFFPCTGTTNKCKPYATQLAELLDTISSQKLAIRRIWLDIETDQVCSPFDYGAQGNIAEAKKLVAAFRAAKHDWGIYTSPTQWETIFGSKTFVLANDVPLWFAKFDNVETLDLKTPFGGWTKADAKQYTDQSASKKFDLNVFSA

SEQ ID NO: 27 - protein, 207 aa, Lecanicillium sp. WMM742
SVQGFDISHYQSSVNFGAAYADGARFVIIKATEGTTYRDPKFSEHYGGATKAGFIRGGYHFAQPASSSGAAQANFFLAHGGGWSGDGITLPGMLDLEYGPNGNTCYGLGPASMRSWISDFVETYHAKTGRYPLIYTSTSWWKTCTGNTSLFADKCPLVVARYNSQVGELPAGWGFYTFWQFNDHYKHGGDSDVFNGAYSQLQKIATG

SEQ ID NO: 28 - protein, 208 aa, Zopfiella sp. t180-6
AVQGFDVSHWQSSVNFAAAYNSGARFVIIKATESNNYIDPKFNTYYPAATSAGLIRGGYHFAHPGETTGAVQADYFIAHGGGWSSDGITLPGMLDLENASGYPACWGLSQSAMVSWIKAFSDRYKARTGVYPMLYTNPSWWTSCTGNSNAFVNTNPLVLARYASSPGTIPGGWPYQTIWQNSDSYTYGGDSDIFNGDLAGLKRLAKGS

SEQ ID NO: 29 - protein, 217 aa, Malbranchea flava
GPKEFESRASGVQGFDISGWQSNVNFAGAYNSGARFVMIKASEGTTFKDRQFSNHYIGATKAGFIRGGYHFALPDVSSATAQVNHFLASGGGWSRDGITLPGMLDIESNPYGAQCYGLDAGRMVAWIREFVDAYKRATGRYPLIYTSPSWWQTCTGNSNAFIDKCPLVLARWASSPGTPPGGWPFHSFWQYADSYQFGGDAQVFNGDEAGLKRMALG

SEQ ID NO: 30 - protein, 208 aa, Hypholoma polytrichi
LVYGVDSSSLVPVATYQKALGEGFTKAVIRGYEEACGVGGEVDPNFVPSYKNARAAGYTDIDMYWFPCNGSTHSCKSYAAQLAAIAAAFSANAMKIGTIWIDIEKDAAICNNWDYGTAGNLAQAKALIAAAKASGFNFGIYSSPGEWSTIFGSTSVVVDNSAPLWFATYNNVQTLTLGTPFGGWSTAVGHQYTDVSASGLFDLNVFAH

SEQ ID NO: 31 - protein, 201 aa, Aspergillus deflectus
AVQGFDISHYQSSVDYAGAYSSGARFVMIKATEGTTYTDPAFSTHYTGATNAGLIRGGYHFARPGSSSGAAQAEYFLAHGGGWTGDGITLPGMLDLEAGCSGLSASAMVSWIQDFGETYKASTGRYPMIYTTTSWWSSCTGNNGGFGDYPLVLARWASSPGELPNGWSVHSFWQNADTYEYGGDSEIWNGSQENLVKFASQ

SEQ ID NO: 32 - protein, 202 aa, Ascobolus stictoideus
AVPGFDISHYQSTVNFADAYSSGARFVIIKATEGTTYKDPKFSSHYTGATNAGLIRGGYHFARPASSTGAVQAQYFVSNGGGWSGDGLTLPGMLDLEGDCAGLSQAGMVSWITSFVNKYKALTTRYPMIYTTNSWWNTCTGNSQAFSANCPLVIARYNSVVGTIPGGWPYYTIWQFNDAYSYGGDSDTFNGAYSQLVKLATG

SEQ ID NO: 33 - protein, 207 aa, Coniochaeta sp
TVQGFDISHYQPTVNYAGAYNAGARFVIIKATEGTTYTDPSFSTHYNGATKAGLIRGGYHFAHPGVTTGAAEANFFLAHGGGWSGDGITLPGMLDLESEGSNPQCWGLSTSGMVAWIKSFSDRYHTVTGRYPMLYTNPSWWSTCTGNSNAFVNTNPLVLARYASAPGTIPGGWPYQTIWQNSDSYSYGGDSDIFNGNLASLQKLATG

SEQ ID NO: 34 - protein, 202 aa, Daldinia fissa
AVPGFDISHYQSSVDFGAAYSSGARFVIIKATEGTTYQDPKFSSHYAGATNAGLIRGGYHFARPASSSGAAQATFFLAHGGGWSGDGITLPGMLDLEGDCAGLSTSAMVSWIRDFSDTYHGKTGRYPLLYTNPSWWSSCTGGSSAFVNTNPLVLARYASSPGALPGGWPYYTIWQFNDAYKYGGDSDTFNGDLTQLKKLASG

SEQ ID NO: 35 - protein, 201 aa, Rosellinia sp
AVQGFDISHYQSSVNFAGAYSAGARFVIIKATEGTSYIDPKFSSHYIGATNAGLIRGGYHFAHLGSSSGAAQANYFLAHGGGWSGDGITLPGMLDLEGDCVLSASGAVAWIKDFSDTYHSKTGVYPLLYTNPSWWSSCTGNSNAFVNTNPLVLARYSSSAGTPPGGWPYYTIWQYNDAYAYGGDSDVFNGDMAGLLRLAKG

SEQ ID NO: 36 - protein, 202 aa, Ascobolus sp. ZY179
AVPGFDISHWQSSVNFASAYSSGARFVIIKATEGTTYKDPKFSSHYTGATKAGFIRGGYHFARPASSTGAAQAQFFASNGGGWSGDGITLPGMLDLEGDCAGLSQSGMVSWISSFVNKYRSLTGRYPMIYTTNSWWVTCTGNSKAFSSNCPLVIARYNSVVGTIPGGWPYYTIWQYNDAYKYGGDSDTFNGAYSQLVKLATG

SEQ ID NO: 37 - protein, 206 aa, Curreya sp. XZ2623
TVPGFDISHYQGTVNFAGAYSSGARFVIIKATEGTTYTDPNFSNNYVGATNAKFIRGAYHFARPDGGSGSTQAQFFHSHGGGWSGDGITLPGMLDIEYGPTSTCYGLSTSAMVTWITDFVNEYHALTGRYPLIYTTNDWWNTCTGNTNKFSTTCPLVLARYSSSVGTIPGGWPFQTIWQFNDNYAYGGDSDTFNGDLAGLKKLATG

SEQ ID NO: 38 - protein, 202 aa, Coniothyrium sp
AVQGFDISHYQASVNFAAAYSGGLRFVYIKATEGTTYQDPAFSSHYSGATSAGFIRGGYHFARPASSTGAAQASYFVAHGGGWSNDGITLPGMLDLEGDCAGLSTASMVSWISSFSNQYHSLTGRWPVIYTTNSWWTTCTGNSAAFNANSPLMLARWGSTAGTIPGGWPYYTIWQYKDSNTYGGDSDVFNGDATQLKKLATG

SEQ ID NO: 39 - protein, 202 aa, Hypoxylon sp
AVPGFDISHYQSNVDFGAAYSSGARFVIIKATEGTTYQDPSFSTHYTGATKAGLIRGSYHFARPGSSSGAAQATYFLAHGGGWSGDGITLPGMLDLEGDCAGLSTNAMVAWIRDFSDTYHGRTGRYPLLYTNPSWWSGCAGGSAAFVGTNPLVLARYAGSPGALPGGWPYYTIWQFDDAYKYGGDSDTFNGDLTQLKKLASG

SEQ ID NO: 40 - protein, 202 aa, Xylariaceae sp. 1653h
AVPGFDISHYQSSVDFAAAYSAGARFVIIKATEGTTYIDPSFSSHYTGATNAGLIRGGYHFAHPGSSSGATQANYFLAHGGGWSGDGITLPGMLDLEGDCAGLSTSAMVSWIKDFSNAYHSKTGRYPLLYTNPSWWSSCTGSSSAFVNTNPLVLARYSSSAGTPPGGWPYYTIWQFNDAYKYGGDSDTFNGEYASLQKLATG

SEQ ID NO: 41 - protein, 202 aa, Hypoxylon sp
AVPGFDISHYQSSVDFAAAYNSGARFVIIKATEGTTYIDPSFSSHYTGATKAGFIRGGYHFAHPGSSSGAAQANYFLAHGGGWSGDGITFPGMLDLEGDCAGLSTSAMVSWIKDFSDTYHSKTGRYPLLYTNPSWWSSCTGDSSAFVNTNPLVLARYSSSAGTPPGGWPYYTIWQFNDAYKYGGDSDTFNGNYASLQKLATG

SEQ ID NO: 42 - protein, 206 aa, Yunnania penicillata
DVDGFDISHYQETVDYAGAYGAGARFVIIKATEGTNYIDSSFNTHYAGATDAGLIRGGYHFAHPGETTGAEQADYFIAHGGNWSNDGITLPGMLDLEGEGSTTCWDLSAADMVAWIKAFSDRYQEVTSRYPLLYTNPSWWSECTGNSDAFVDTNPLVLARYASSPGEIPGGWPAQTIWQNSDSYSFGGDSDIFNGDEAGLKKLASG

SEQ ID NO: 43 - protein, 207 aa, Engyodontium album
RVQGFDISHYQPSVDFNAAYADGARFVIIKATEGTTYKDPKFSQHYIGATNAGFIRGGYHFAQPASSSGAAQADYFLKNGGGWSSDGITLPGMLDMEYNPNGSACYGLSQASMRNWINDFVNTYHSRTGVYPLLYTTTSWWKTCTGNTAMFADKCPLVIARYNSVVGELPAGWSFWTIWQYNDHYKHGGDSDAFNGDYSQLQRIARG

SEQ ID NO: 44 - protein, 208 aa, Metapochonia bulbillosa
TVAGFDISNYQPTVDFKKAYADGARFVIIKATEGTTYTDPSFSSHYTGATQAGLIRGGYHFAHPGSGTGAAQANYFLAHGGGWSKDGITLPGMIDLEYNPSGATCYGLSASGMVSWISDFVETYHSKTGVYPLIYTSTSWWNQCTGSSTAFGNKCPLVVARYASSVGALPAGWGFQTIWQNSDKSPWGGDNDIFNGSLDQLKRIANAS

SEQ ID NO: 45 - protein, 215 aa, Hamigera paravellanea
APLEARAGSVQGFDISHYQAKVDFAAAYRSGARFVIIKATEGTTYTDPAFSSHYTSATNAGFIRGGYHFAHPDSSSGAAQATYFLAHGGGWSGDGITLPGMLDLEYNPSGATCYGLSDAAMVAWIQDFVDTYHARTGRYPMIYTTADWWNTCTGNSSKFSQTCPLVLARYASSVGTVPGGWGYQTIWQNSDSYAYGGDSDIFNGDETQLKKLASG

SEQ ID NO: 46 - protein, 217 aa, Metarhizium iadini
SPVELEQRAASVKGFDISGYQPNVDFNKAYADGARFVIIKATEGTTYIDKTFSKHYTGATKAKLIRGAYHFAHPGQNKASAEADFFVQHGGNWSKDAITLPGMVDLESEKGHPPCWGLSHSAMVAWISEFVAAYHKKTTRYPMLYTNPSWWSACTGNSKAFKDTCPLVLARYASSPGAIPGGWPAQTIWQNSDKSPWGGDSDMFNGDLAQLKKLATG

SEQ ID NO: 47 - protein, 214 aa, Thermoascus aurantiacus
ELDKRARGVQGFDISHYQPNVDFKGAYNSGARFVIIKATEGTTYKDPAFSKHYIGATEAGLIRGGYHFAHPDKSSGAAQANFFLAHGGGWSGDGITLPGMVDLEYNPSGDACYGLSDSQMVSWIRDFVNTYHAHTGRYPMIYTTADWWKRCTGDSHAFSTTCPLVLARYNSSPGTVPGGWPYHTIWQNSDKYRFGGDSDIFNGDLAGLKRLAKG

SEQ ID NO: 48 - protein, 208 aa, Clonostachys rossmaniae
AVPGFDISGWQKSTDFAKSYANGDRFVYIKATEGTTFKNPLFSKQYTGATNARLIRGAYHFAQPASSSGASQARFFVANGGGWSNDGITLPGAVDMEYNPSGATCYGLSKTAMVNWIEDFVSTYQALTGRWPVVYTTLDWWTQCTGNSAKFGDRCPLWVARYASAVGQIPAGWSFHTIWQYNAKYPEGGDSDIFNGDETRLKALASGA

SEQ ID NO: 49 - protein, 203 aa, Simplicillium obclavatum
APKGIDVSHWQGSINWGAVKANGIEWAYIKATESTNYKDPNFNANYVGATNAGLIRGAYHFARPGDSSGAAQANYFASNGGGWSADGITLPGAVDLEAGCSGLSQSAMTAWIKDFSNTYHARTGRYPAIYTTTSWWKQCTGNASGFQNNNPLWIARWASSVGELPAGYSYHTFWQYADHGPNPGDQDVFNGDSAGLKRMAKGS

SEQ ID NO: 50 - protein, 216 aa, Aspergillus inflatus
APLEARANTVQGFDISSFQPNVDFAAAYKAGARFVMMKATQNTNYVDKTFNAHYEGATKAGLIRGGYHFAIPNGPSGAAQAEYFLAHGGGWSDDGKTLPGMIDLEYNPYGQTCYDLSAAKMVDWIKDFSNTYHAKTKRYPMIYTTANWWKECTGDSKEFSQTNPLVLARYSSSAGTVPGGWPAYSFWQNADKYKFGGDSDIWNGSEDNLKKFAKGA

SEQ ID NO: 51 - protein, 207 aa, Paracremonium inflatum
KVLGFDISHYQATVDFNAAKDAGARFVIIKATEGTTYKDPAFSKHYTGATKAGLIRGGYHFAQPASSSGAAQATFFLAHGGGWSSDGITLPGMLDLEYNPSGSTCYGLSQSSMVQWISDFIDTYHSKTGRYPLIYTSTSWWKTCTGNSSKFAANCPLVVARYSSSVGELPAGWTYYTIWQNSDSYKYGGDSDIFNGDESQLQKLAKG

SEQ ID NO: 52 - protein, 208 aa, Westerdykella sp
AVSGMDISHYQGTNYNFAGAYSSGARFVIIKATEGTTYTDPQFSANYIAATNAGFIRGGYHFARPADSTGAAQAKYFVSHGGGWSSDGITLPGMLDLEYGSSSACHGLSVSAMNTWIASFINQYRSLTGAYPMIYTTADWWKTCTGDSQAWNTKCPLVLARYSSSVGTIPGGWPYQTIWQFNDSYKYGGDSDTFNGDLAGLKRLAKGS

SEQ ID NO: 53 - protein, 207 aa, Stropharia semiglobata
LTYAVDSSTLVSVATYTKAKSQGFTKAIIRGYQEACGSGGAVDPNFVQTYKNARAAGYTDIDMYWFPCNGSTHNCKSYATQIAAIAATFSANSMKIGRIWIDIEKDAAVCNNWNYGTAGNLSQAKALISAIKASGFVYGIYSSPGEWGNIFGSTSVVVDNSAPLWFATWNNVQTLTMGTKFGGWTSAMGHQYTDVSASGQFDLSVFA

SEQ ID NO: 54 - protein, 208 aa, Gelasinospora cratophora
TVQGFDISHYQSSVNFAGAYSSGARFVIIKATEGTTYIDSSFSSHYTGATSAGLIRGGYHFAHPDSSTGAAQADYFLAHGGGWSADGITLPGMIDLESVSGKATCFGLSTSAMVSWIKSFSDRYYAKTGRYPMIYTNYSWWNQCTGNSASFAATNPLVLARWSSTVGTLPGGWSVQTIWQNADTYTYGGDSDVFNGSLDRLKALAKGS

SEQ ID NO: 55 - protein, 207 aa, Flammulina velutipes
RLNGIDVSGYQPNVNWATVKANGVSFAYIKATEGTTYTNPSFSSQYTGATKAGLIRGSYHFAHPSSSTGAAQARYFVAHGGGWSGDGITLPGALDIEYNPSGATCYGLSTSSMVNWIADFSNTYHSLTGRYPVIYTTADWWRTCTGNSASFANNSPLWIARYASTIGTLPAGWSYATFWQYADSGSNPGDQDYFNGDAAGLKRLATS

SEQ ID NO: 56 - protein, 207 aa, Deconica coprophila
LVHAVDSSSLVSTATFSKAKSEGFTKAVIRGYQEACGSGGRVDPNFVQTYKNARAAGITNIDTYWYPCNGSGNSCKSYAKQIAGISATFNAHSMKIGRIWIDIEKDSICNNWNYGTSGNRDHAKKLITAIKNSGFKYGIYSSPGEWSTIFGSESFDLDSGAPLWFATWNNVQTLTLGTHFGGWTSAHGHQYTDKSASGQFDLNVFSS

SEQ ID NO: 57 - protein, 208 aa, Rhizomucor pusillus
YETGVDVSALTSTSAWSCAKKLGYDHAIVRCYIEAYGGNPGGKIDSNCFQNYKNAKAGGFTSVDIYMFPCTGRSTCKSPAAQVKEVVDYVGSNKMTVGRLWLDVEIDPSANNWPSASSARSTLKSFKSALDSTGWKYGIYSSASQWSQITGSSSWELDSSLPLWYAHYDASLSFSDFSPFGGWTKPTIKQYAGSVSFCSAGWDKNYYG

SEQ ID NO: 58 - protein, 207 aa, Stropharia semiglobata
LVYGVDSSTLVSTATYSKAKSEGFTKAIIRGYQEACGSGGRVDPNFVATYKNARAAGITDIDMYWFPCNGSGNSCKSYAKQLSEIANVFSANSMKIGTIWIDFEKDSGCNNWNYGTTGNLNHAKALISAIKATGFKFGIYSSPGEWGTLFGSTGVVLDSSAPLWFATWNNVKTLTLGTHFGGWTKAVGHQYTDVSASGQFDLNVFAN

SEQ ID NO: 59 - protein, 207 aa, Stropharia semiglobata
LVYGVDSSTLVSTATYKKAKSEGFTKAIIRGYQEACGSGGRVDPNFVATYKNARAAGITDIDMYWFPCNGSGNSCKSYAKQLSEIANVFSANSMKIGTIWIDFEKDSGCNNWNYGTTGNLNHAKALISAIKATGFKFGIYSSPGEWGTLFGSTGVVLDSSAPLWFATWNNVKTLTLGTHFGGWTTAAGHQYTDVSSSGQFDLNVFAN

SEQ ID NO: 60 - protein, 207 aa, Myceliophthora fergusii
AVQGFDISHWQSSVDFKAAYNSGARFVIIKATEGTSFIDPKFSSHYTGATNAGFIRGAYHFAHPGQSSGEAQADYFLAHGGGWTPDGITLPGMLDLEAYNAGECWGLSQSAMVAWIKAFSDRYHARTGVYPMLYTNLSWWKTCTGNSKAFVNTNPLVLARWASSPGEIPGGWPWQTIWQNSDSYRYGGDSDIFNGDMNQLRRLATAA

SEQ ID NO: 61 - protein, 204 aa, Mortierella alpina
ALPKGIDVSHWQGDVNWNSVKAAGIEFVYIKATESINYIDSKFDANYVGATNAGLIRGGYHFARPAASSGAVQANYFLANGGGWSSDGITLPGALDLEAGCSGLSQAAMTAWVRDFSDTYHARTGRYPVIYTTTSWWKQCTGNASGFQNNNPLWIARWASSAGELPAGYAFHTFWQYADKGPNPGDQDYFNGDSAGLRRFAKGS

SEQ ID NO: 62 - protein, 216 aa, Penicillium atrovenetum
TPLESRASGVQGFDISSYQGTVDFAGAYAAGARFVMIKATEGTTYTDKTFSSHYEGASSAGLIRGGYHFAHPDSSSGAKQAEYFLAHGGGWSNDGKTLPGMLDIEYNPSGATCYGISKSAMVAWVKDFGETYKGKTGRYPMIYTTADWWNTCTGGSTAFSKDYPLVLARYSSSVGTIPGGWPYQSFWQNSDKYTYGGDSDLWNGSEASLKTFAKGA

SEQ ID NO: 63 - protein, 245 aa, Trichophaea saccata
YPVKTDLHCRSSPSTSASIVRTYSSGTEVQIQCQTTGTSVQGSNVWDKTQHGCYVADYYVKTGHSGIFTTKCGSSSGGGSCKPPPINAATVALIKEFEGFVPKPAPDPIGLPTVGYGHLCKTKGCKEVPYSFPLTQETATKLLQSDIKTFTSCVSNYVKDSVKLNDNQYGALASWAFNVGCGNVQTSSLIKRLNAGENPNTVAAQELPKWKYAGGKVMPGLVRRRNAEVALFKKPSSVQAHPPKC

SEQ ID NO: 64 - protein, 249 aa, Chaetomium thermophilum
PASAYAITGDNVNCRSGPGTSYAVKKVYKKGTDVKISCQTTGTNINGNNLWDKTSDGCYVSDYYVKTGSNGYVTSKCSSSGGSTCAAPKSNQATVDLIAEFEGFRANIYTDAAGYATVGYGHKCQKAKCAEVKYKIPLSKADGKKLLADDMRSFEVCITNMLNSKAKLNYNQFGALVSWSFNVGCGAAKSSTLIKRLNNGENVNKVLSEELPKWNKAGGKVLQGLVRRRAAEVALAKKSGSSQALPVKC

SEQ ID NO: 65 - protein, 248 aa, Trichoderma harzianum
YPITGDVVNCRTGPGTSYAIKKSYKKNQDISISCQTAGTSVNGNSIWDKTADGCYVADYYVKTGSSGYVTKKCTASSGGGSSSSYCKTINSAGVDLIAKWEGFVASPKPDPIGLPTVGYGHLCQQKNCREVKYKFPLTKTTAKELLLDDLPKYTKCLADYLNDKPKLNANQWAALTSWVFNVGCGNAKTSTLVKRLNNGEAANTVAAEELPKWRMAGGKVLPGLEARRKDEVKLFKTASSKQAYPKCQ

SEQ ID NO: 66 - protein, 245 aa, Trichophaea minuta
YPAKVDLRCRSSPSTSASVVRTYSKGSEIQISCQTTGTSVEGSNVWDKTQHGCYVADYYVKTGHSGIFTTKCGSSSGGGSCKPPPINAATVALIKEFEGFVAKPAPDPIGLPTVGYGHLCKTKGCKEVPYSFPLTQTTATKLLQSDIKTFTSCVSNYVKDSVKLNDNQFGALSSWAFNVGCGNIQTSSLIKRLNAGENPNTVAAQELPKWKYAGGKVLPGLVRRRKAEVALFKKPSSVQAHPPKC

SEQ ID NO: 67 - protein, 249 aa, Chaetomium sp. ZY287
YKISGSSVNCRSGPGTNYPVKKTYANGDEVTISCQTTGTNVEGNNIWDKTQHGCYVADKYVKTGKDGFVTKKCGSSGGGGGGKTCKAPKSNAATVDLIASFEGFRANIYTDATGHPTVGYGHMCTKSRCAEVKYKIPLSKADGKKLLADDMAKFEKCIKEMLNSKAKLNLNQYGALVSWSFNVGCGAAKGSQLVSRLNKGENPNTVLSNELPKWVHGNGKVLPGLVRRRNAEIALAKKSGSGAALPVKC

SEQ ID NO: 68 - protein, 245 aa, Mortierella sp. ZY002
YPITGADALHCRSGPGTSYPIQKTLRPPQDIKIQCQEPGTVVNGVSLWDKTQFGCYVSDYYVKTGTGNYVAPRCNSGGSSSACTGLNDAGINLIKEFEGFVPRPAPDPIGLPTVGYGHLCQTKGCGEVKYSFPLTTATATALLKDDLPKYTSCLAKALNGKPKLNKNQWAALASWTFNVGCGNMKSSSLITRLNAGQNPNTVATEELPKWKLAGGKVLPGLVRRRAAEVKLFKTANSSQGYPKCA

SEQ ID NO: 69 - protein, 247 aa, Metarhizium sp. XZ2431
YPVSADSLNCRAEPNTSSAIKTTYKKGEDVKISCQTEGPSINGNTIWDKTQDGCYVADYYVKTGSSGYVTGKCGGSSPPSGSGFCKTVNKAGLDLITKWEGFVSSPRGDPIGLPTVGYGHLCQKKGCAEVKYKFPLTKATALQLLNDDLPKYTGCLGKLLNSKVKLNDNQWAALTSWVFNVGCGNAQSSSLVRRLNNGENPNTVAPSELPKWKMAGGKVLEGLVKRRADEVRLFKVSSSKGAFPKCQ

SEQ ID NO: 70 - protein, 250 aa, Geomyces auratus
AFPITGSTVNCRTGPGTSHGVKTSYKKGHEVTVSCQTGGTSVNGNSIWDKTSDGCYVADYYVKTGSSGYVKPKCGSSSGGGGGSSCGAPKSNAATVNLIAEFEGFVSHVYTDATGHPTVGYGHLCSNSKCSGIGYSIPISKANAKKLLAKDMAIAEKCITAMINKSRTLNLNQYGALVSWAFNEGCGAAKSSTLIKRINNGEKPSTVIPQELPKWVYGGSSVLPGLVRRRNAEIALAKKATSSKALPAHC

SEQ ID NO: 71 - protein, 240 aa, Ilyonectria rufa
YKITGDNVNCRSGPGTSYSVKRSFKKGTDVTLSCQTTGENVLGTSIWDKTSYGCYVSDYYVKTGSSGFVVKKCGTCGAPKSNAATVNLISDFEGFRANIYKDAAGYPTVGYGHLCSNSRCTDVPYSIPLSKANGKNLLATDMTKFEKCITAMVSSSVTLNKNQYGALVSWAFNMGCGATKTSTLIKRLNQGQNVNTVLSTELPKWVYAGGKKLNGLVRRRNAEIALAKKKTTEKALPNKC